(19) United States
(12) Patent Application Publication
(10) Pub. No.: US 2010/0324058 A1
(43) Pub. Date: Dec. 23, 2010

(54) THROMBIN FUNCTION COMPOUNDS AND PHARMACEUTICAL COMPOSITIONS BASED ON THEM

(75) Inventors: Elena Ivanovna Sinauridze, Moscow region (RU); Fazoil Inoyatovich Ataullakhanov, Moscow (RU); Andrey Alexandrovich Butylin, Moscow (RU); Vladimir Borisovich Sulimov, Moscow (RU); Alexey Nickolayevich Romanov, Moscow (RU); Alexey Alexeevich Bogolyubov, Tverskaya region (RU); Yury Vladimirovich Kuznetsov, Moscow region (RU); Irina Vladimirovna Gribkova, Moscow (RU); Alexander Sergeevich Gorbatenko, Moscow (RU); Olga Anatolievna Kondakova, Moscow (RU)

Correspondence Address: KNOBBE MARTENS OLSON & BEAR LLP, 2040 MAIN STREET, FOURTEENTH FLOOR, IRVINE, CA, 92614, US

(73) Assignee: Obschestvo S Organichennoi Otvetsttvennoctiyou "Bionika", Moscow (RU)

(21) Appl. No.: 12/666,221

(22) PCT Filed: Jun. 27, 2008
(86) PCT No.: PCT/RU2008/000400

§ 371 Date: Dec. 22, 2009

(30) Foreign Application Priority Data
Jun. 28, 2007 (RU) 2007124201

(51) Int. Cl. A61K 31/4425; C07D 213/73; C07D 277/40; A61K 31/426; C07C 309/76; A61K 31/255; C07D 401/06; A61K 31/4439; C07D 401/12; A61K 31/444; A61K 31/506; A61K 31/497; A61K 31/501; A61K 31/4545; A61P 7/02

(52) U.S. Cl. 514/252.03; 546/311; 514/352; 548/199; 514/370; 558/58; 514/518; 546/273.4; 514/338; 546/261; 514/335; 544/319; 514/269; 544/405; 514/255.05; 544/238; 546/194; 514/318

(57) ABSTRACT

This invention relates to new chemical compounds, application of these compound as thrombin inhibitors, and pharmaceutical compositions based on them, and can be used to treat and prevent thrombin-dependent thromboembolic events, and in research.

This invention relates to new chemical compounds, application of these compounds as thrombin inhibitors, and pharmaceutical compositions based on them, and can be used for treating and preventing thrombin-dependent thromboembolic events, and for research purposes.

Thrombin is the principal enzyme of the blood clotting system converting the soluble plasma protein, fibrinogen, into an insoluble fibrin clot. A fragile equilibrium exists between thrombin formation, a process that causes fibrin polymerization, and thrombin inhibition, that is, a process that suppresses thrombin activity. Excessive thrombin formation results in thromboses.

Direct thrombin inhibitors is the name for inhibitors that are strongly bound directly to the active enzyme center and block fibrinogen, a natural substrate, off the active center. This blockage halts thrombin-catalyzed fibrin conversion from fibrinogen and, as a result, prevents fibrin clotting and slows down blood clotting or prevents its completely. To have strong antithrombin activity, therefore, direct thrombin inhibitors are to combine with a maximum possible strength with the active thrombin center. For this purpose, they are to meet several conditions dictated by the structure of the active center of a thrombin molecule.

The active thrombin center is commonly divided, for convenience, into several cavities, or pockets, to receive different amino acids of its fibrinogen substrate near the point where an amidolytic reaction takes place. Pocket S1 is a deep and narrow cavity with walls formed by hydrophobic amino acid residues and, actually on the bottom of the cavity, a negative charge source created in the presence of the carboxyl group of amino acid Asp 189. Pocket Si serves to bind the principal amino acid residues (lysine or arginine) in fibrinogen directly at the breakup point of the peptide bond (at the C-end of lysine or arginine). The long unbranched hydrocarbon residue of the principal amino acid extends the full length of pocket S1, while the positively charged main fragment at the end of the hydrocarbon residue forms a salt bridge to the negatively charged aspartic residue at the bottom of pocket S1. Pocket S1 is, therefore, best suited for identifying principal amino acid residues in the polypeptide chain of fibrinogen.

Another pocket, S2, formed by non-polar amino acid residues, adjoins immediately pocket Si and serves to identify minor hydrophobic amino acids (valine, isoleucine, and leucine) in the amino acid sequence of fibrinogen behind the principal amino acid received in pocket S1 (at the N-end of the principal amino acid). Pocket S2 has a slightly smaller volume than pocket S1, and it does not contain any charged amino acid groups. Pocket S2 is, therefore, ideally suited for binding small hydrocarbon residues of non-polar aliphatic amino acids.

Yet another pocket, S3, is found next to pocket S2 on thrombin surface. This is also a hydrophobic pocket, but it has a rather large volume and is not precisely defined, because a considerable part of it is open and exposed directly to the solvent. Pocket S3 serves to receive large aliphatic and aromatic hydrophobic amino acid fragments of fibrinogen two or three links away from the break in the peptide chain.

A direct thrombin inhibitor must fill in an optimal manner these three pockets of the active center of a thrombin molecule. For example, the well-known tripeptide inhibitor D-Phe-Pro-Arg was found by X-ray structure analysis to react with the active thrombin center as follows: the arginine residue fills pocket S1, the proline residue takes up pocket S2, and D -phenylalanine occupies pocket S3.

Medications used in current clinical practice to control thromboses are not always suited for inhibiting excess thrombin already formed in blood. Doctors tend to liberally use indirect thrombin inhibitors, such as unfractionated heparin and low molecular weight heparins, and vitamin K antagonists (warfarin). All these medications cannot by themselves inhibit excess thrombin accumulating in the system. Various heparins only accelerate the inhibiting effect of the natural thrombin inhibitor—antithrombin III (AT III)—present in plasma, and so heparins have only a weak anticoagulant effect if the AT III content in the patient's plasma is very low for one reason or another. Vitamin K antagonists reduce the clotting rate by suppressing syntheses of the precursors of clotting factors in the liver. Obviously, this is a relatively slow option that cannot help in serious situations requiring quick suppression of thrombin present in the blood.

The restrictions of indirect coagulant therapy have led to attempts by pharmaceutical companies to develop a potent and selective direct thrombin inhibitor. By now, a large number of such thrombin inhibitors has been developed. A majority of them do not, however, exhibit all the properties required of a drug. Research continues to improve their pharmacological properties such as effective time, low toxicity, solubility in water, oral bioavailability, and so on. An ideal thrombin inhibitor must be effective against thrombin fixed in the clot as well. It must be selective to thrombin without inhibiting the proteases involved in fibrinolysis, remain in the blood for a long time, resist the effect of enzymes and cytochrome P450 in the liver, be kept in an aqueous medium, immune to combining (or combining only slightly) with blood proteins, and be nontoxic. Preliminary testing of a compound, however, is inconclusive about its suitability in meeting these requirements. Even though a large number of effective low molecular weight thrombin inhibitors has been synthesized already, only one, Argatroban synthesized in Japan (U.S. Pat. No. 5,214,052, 1993), which has passed all necessary clinical tests, is used today. It is not, however, an ideal inhibitor, because it has a low stability in solutions (its T_{1/2} in plasma is 36 minutes). Which means that the need for developing new effective and safe synthetic thrombin inhibitors continues to present a challenge.

Published patents and scientific studies available today describe a large number of thrombin inhibitors. A summary of these publications follows below:

U.S. Patent Application No. 2006/0014699 (Astra Zeneca AB), 2006, and U.S. Pat. No. 5,795,896 (Astra Aktiebolag), 1998, describe antithrombotic pharmaceutical compounds containing melagatran inhibitor.

Also known in the art are pyrrolidine thrombin inhibitors described in U.S. Pat. No. 5,510,369 (Merck & Co), 1996, and pyridine thrombin inhibitors, such as those described in U.S. Pat. No. 5,792,779 (Merck & Co), 1998.

This applicant has studied many scientific papers and articles containing information about the structure of existing inhibitors and the mechanism of reaction between the inhibitor and a thrombin molecule. The publications studied, as shown in Table 1, cover virtually all classes of chemical compounds known as thrombin inhibitors. The list of publications appearing in Table 1 is full enough, if far from complete. As we developed our own thrombin inhibitors we deliberately avoided structures described in these publications. The publications we refer to do not contain information about thrombin inhibitors having elements characterizing the new compounds we claim as inventions.

The practical task of this invention is developing new compounds that could serve as direct thrombin inhibitors. These inhibitors can be used to treat acute thrombotic conditions developing in the organism under the effect of various pathologies. An enormous number of different pathological conditions of the organism is related to disorders in the hemostatic system. Thromboembolic complications arising as a result of diseases such as myocardial infarction, stroke, thrombosis of deep veins or pulmonary artery, are among the primary causes of death around the world. Little surprise then that intensive efforts have been going on for years to develop medications that could serve as effective and safe clinical drugs. Above all, these are antithrombotic agents displaying anticoagulant properties.

Unless indicated otherwise, the following definitions are used in this description:

Active center is an area of the protein macromolecule that plays a key role in biochemical reactions.

Protein means a protein macromolecule.

Target protein means a protein macromolecule involved in the binding process.

Ligands means collections of low molecular weight chemical structures.

Binding process means formation of Van der Waals' or a covalent complex of a ligand and the active center of the target protein.

Screening means identification of a set of compounds in a collection of chemical structures that react selectively with a specific area of the protein macromolecule.

Correct positioning means positioning to place a ligand in a position corresponding to the minimum free energy of the ligand-protein complex.

Selective ligand means a ligand that is bound in a specific manner to a particular target protein.

Validation means a series of calculations and comparison methodology to assess the quality of the system in operation and its efficiency in selecting ligands from a random set of ligands that are bound reliably to a given target protein.

Reference protein means a protein used to either adjust the parameters of a model calculation (score) in accordance with experimental data, or during validation of the operating system, or to assess the binding specificity of a particular inhibitor.

Specifically binding ligand means a ligand that is bound to a particular protein only, but not to any other proteins.

Inhibitor means a ligand that is bound to the active center of a particular target protein and blocks the normal course of biochemical reactions.

Docking means the positioning of a ligand in the active center of a protein.

Scoring means calculation to assess the free energy needed to bind a ligand to a protein.

ΔG binding means the resulting free energy calculation gain needed to bind a ligand to a target protein (using the SOL software).

C₁₋₆ alkyl means an alkyl group comprising an unbranched or branched hydrocarbon chain with 1 to 6 carbon atoms, for example, methyl, ethyl, n-propyl, isopropyl, n-butyl, isobutyl, tert-butyl, and so on.

C₁₋₆ alkoxy means an alkoxy group containing an unbranched or branched hydrocarbon chain with 1 to 6 carbon atoms, for example, methoxy, ethoxy, n-propoxy, isopropoxy, and so on.

Halogen means chlorine, bromine, iodine, or fluorine.

Pharmaceutically acceptable salt means any salt produced by an active compound of formula (I), unless it is toxic or inhibits adsorption and pharmacological effect of the active compound. Such salt can be produced by reaction between a compound of formula (I) and an organic or inorganic base, such as sodium hydroxide, potassium hydroxide, ammonium hydroxide, methylamine, ethylamine, and the like.

Solvate means the crystalline form of an active compound of formula (I) whose crystalline lattice contains molecules of water or another solvent from which the active compound of formula (I) has crystallized.

Pharmaceutically acceptable carrier means a carrier that must be compatible with the other ingredients of a composition and be harmless to the recipient, that is, be nontoxic to cells or mammals in doses and concentrations in which it is used. Frequently, a pharmaceutically acceptable carrier is an aqueous pH buffering solution. Examples of physiologically acceptable carriers include buffers such as solutions based on phosphates, citrates, or other salts of organic acids; antioxidants including ascorbic acid, polypeptides of low molecular weight (less than 10 residues); proteins such as serum albumin, gelatin or immunoglobulins; hydrophilic polymers such as polyvinyl pyrrolidone; amino acids such as glycine, glutamine, asparagine, arginine, or lysine; monosaccharides, disaccharides, and other carbohydrates, including glucose, mannose or dextrins; chelating agents such as EDTA; and sugar alcohols such as mannitol or sorbitol.

Therapeutically effective quantity means a quantity needed to achieve the desired extent of thrombin inhibition in a mammal organism.

Mammal, in the sense in which it is used here, include primates (for example, humans, anthropoid apes, non-anthropoid apes, and lower monkeys), predators (for example, cats, dogs, and bears), rodents (for example, mouse, rat, and squirrel), insectivores (for example, shrew and mole), and so on.

The practical task set by the applicant is achieved by developing a compound of general structural formula (I), including its pharmaceutically acceptable salts or solvates:

A-B-C (I)

wherein C is chosen from a group containing the following structures:

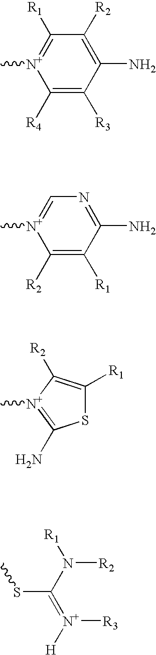

wherein R₁, R₂, R₃, and R₄ are, independently from one another, hydrogen or C₁₋₆ alkyl;

B —(CH₂)ₙ—, wherein n is an integer from 1 to 5; and

A is selected from a group containing the structures:

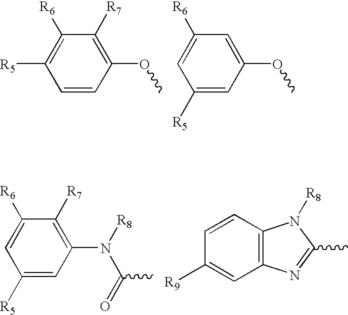

wherein R₅ is selected from a group containing hydrogen, C₁₋₆-alkoxy, CH₂NR₁₀R₁₁, and CH(CH₃)NR₁₀R₁₁,

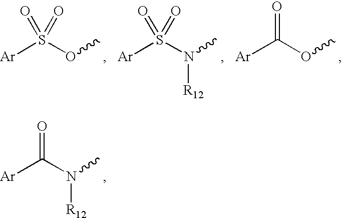

wherein R₆ and R₇ are, independently from each other, hydrogen, C₁₋₆ alkyl; C₁₋₆ alkoxy; or halogen;

R₈ is hydrogen or C₁₋₆ alkyl;

R₉ is chosen from the following group consisting of:

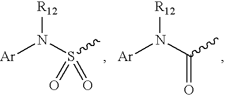

R₁₀ and R₁₂ are independently from each other, selected from a group consisting of hydrogen, C₁₋₆ alkyl; (CH₂)ₘCOOR₁₃, and (CH₂)ₘCON(R₁₃)₂,

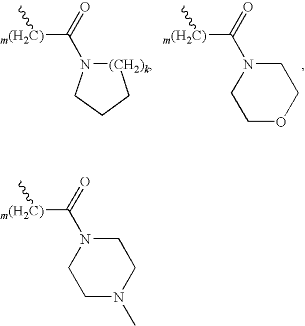

wherein m is an integer from 1 to 4,

R₁₃ is hydrogen or C₁₋₆ alkyl,

R₁₁ is C₁₋₆ alkyl or Ar;

Ar is phenyl, pyridyl, oxazolyl, thiazolyl, thienyl, furanyl, pyrimidinyl, pyridazinyl, pyrazinyl, indolyl, benzofuranyl, or benzothiophenyl having from one to five substituents selected from the group of:

hydrogen, C₁₋₆ alkyl, C₁₋₆ alkoxy, halogen, N(R₁₃)₂, OH, NO₂, CN, COOR₁₃, CON(R₁₃)₂, and SO₂R₁₃.

With the exception of:

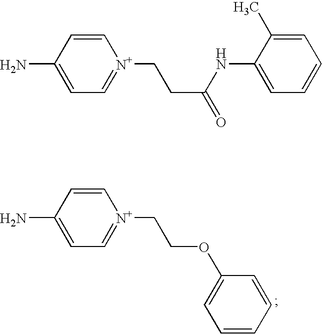

The compounds excluded from this list are already known, in particular, 4-amino-1-[3-[(2-methylphenyl)amino]-3-oxopropyl]pyridinium chloride is described in the Journal of Medicinal Chemistry, 17(7), 739-744, 1974, in “Carbocyclic Derivatives Related to Indoramin; 4-amino-1-(2-phenoxyethyl)-pyridinium bromide is described in the Journal of Organic Chemistry, 26, 2740-7, 1961, in “Application of Sodium Borohydride Reduction to Synthesis of Substituted Aminopiperidines, Aminopiperazines, Aminopyridines And Hydrazines.” It is worthwhile to note, though, that these sources do not refer to the possibility of the compounds described being used as thrombin inhibitors.

The preferred embodiment of this invention describes the following compounds of claim 1, and their pharmaceutically acceptable salts or solvates:

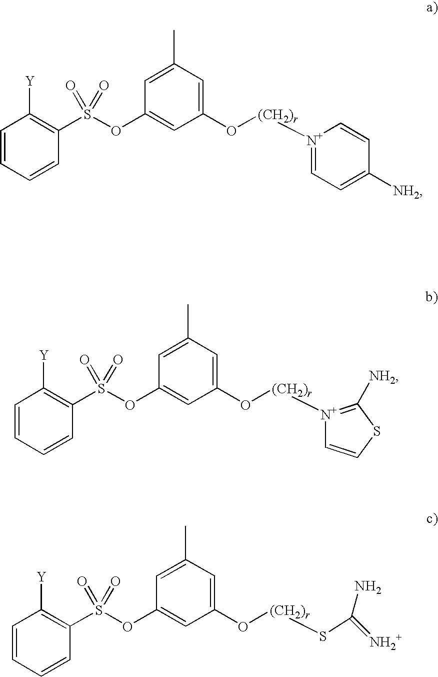

wherein Y is chosen from a group consisting of hydrogen, halogen, COOR₁₃, CON(R₁₃)₂, and SO₂R₁₃; and

r is an integer from 2 to 5.

This applicant has found that a compound of the structural formula A-B-C, and its pharmaceutically acceptable salts or solvates are capable of inhibiting thrombin.

Accordingly, the new compounds and their pharmaceutically acceptable salts or solvates can be used in practice as thrombin inhibitors.

Compounds that could be interesting for practical application as thrombin inhibitors, that is, displaying a significant inhibiting effect, are selected as follows: We constructed three-dimensional models of molecules from a virtual library centered on structures described by general structural formula (I). At the next step, the resulting structures were docked to the active center of a thrombin inhibitor, and the docking results received for the molecular structures of potential thrombin inhibitors were used to select the best prospects, that is, molecules that showed scoring function values (measured in the docking process) not worse that −5.0 kcal/mol. Positioning methods suggested by the docking procedure were visualized for such molecules. If these positioning methods satisfied the above hypothesis regarding inhibitor binding to the active thrombin center, such molecules were considered “virtual hits” and were accepted as prospects for synthesis and experimental measurement of inhibiting activity. The final decision to initiate synthesis was made from an assessment of its probable complexity.

The thrombin inhibitor of this invention meet optimally the above requirement of effective reaction with the active center of thrombin. The positively charged chemical group C of the inhibitor of formula (I) is located at the bottom of pocket S1 forming a salt bridge to the amino acid residue Asp 189. The chemical group B occupies the remaining space of pocket S1 allowing for optimal hydrophobic reaction with the pocket walls. The chemical group A of formula (I) is located in pocket S2, the R groups listed below are hydrophobic fragments, and linkers bonding the separate part of the molecule and exposed to the solvent are located in pocket S3 as well. From the viewpoint of bonding to the active thrombin center, the linkers can be represented by both hydrophilic and hydrophobic molecular groups, but it desirable to partially balance the hydrophobic nature of the inhibitor molecule as a whole by selecting hydrophilic linkers in order to give beneficial pharmaco-kinetic properties to the inhibitor molecule. For this purpose as well, the hydrophobic fragments located in pocket S3 could be modified with hydrophobic residues located in the pocket at the side exposed to the solvent. The thrombin inhibitors described here fully satisfy the above requirements.

This claim is demonstrated by selective positioning (docking) of the thrombin inhibitors of this invention to the active thrombin center following the procedure described below. Docking is effected by global minimization of the total energy of the inhibitor molecule. The total inhibitor energy is comprised of the internal tension energy of the inhibitor in the conformation accounting for inhibitor binding to the active thrombin center and inhibitor energy in the thrombin field. In turn, the thrombin field induces electrostatic, Van der Waals' reaction with the inhibitor molecule, and a number of reactions initiated by solvation and desolvation of individual parts of the thrombin molecule and ligand. These reactions have been described in many publications and are familiar to researchers in this field. Global minimization is repeated several times by using a genetic algorithm. The minimization program results in geometric positioning of the thrombin inhibitor in the active center of this enzyme and a scoring function value that serves as an estimate of the free energy used to form a complex of the thrombin inhibitors described here and the thrombin molecule. For inhibitors described here, the scoring function is always smaller than −5 kcal/mol, which agrees with the inhibition constants in the micromolar range and below. The reliability of prediction using the scoring function can be tested by various methods known to specialists in this field. In particular, the so-called thrombin inhibitor enhancement coefficient showing the selectivity of known active thrombin inhibitors among random molecules on the basis of the scoring function value is equal to 0.85, which is evidence of sufficiently reliable prediction. The geometric positions of the inhibitors described here were achieved by the aforesaid docking procedure and also meet the optimal conditions for binding thrombin inhibitors to the active thrombin center, where their inherent inhibiting activity is displayed in respect of the fibrinogen amidolysis reaction catalyzed by thrombin.

The claimed compounds can be obtained by common methods known to a specialist in organic chemistry.

A great number of various pathological conditions of the organism are related to disorders developing in the hemostasis system. Thromboembolic complications arising in such diseases as myocardial infarction, stroke, thrombosis of deep veins or pulmonary artery are among the chief causes of death around the world.

This invention also includes a pharmaceutical composition for treating and prophylactic prevention of thrombin-dependent thromboembolic events, which comprises a therapeutically effective quantity of the compound of claim 1 or its pharmaceutically acceptable salt or solvate, and a pharmaceutically acceptable carrier.

The compounds of this invention can be administered in any suitable manner leading to their bioaccumulation in blood. This can be achieved by parenteral administration methods, including intravenous, intramuscular, intracutaneous, subcutaneous, and intraperitoneal injections. Other administration methods can be used as well, such as absorption through the gastrointestinal tract by peroral application of appropriate compositions. Peroral application is preferred because of easy use. Alternatively, the medication can be administered through the vaginal and rectal muscle tissue. In addition, the compounds of this invention can be injected through the skin (for example, transdermally) or administered by inhalation. It is to be understood that the preferred method of administration depends on the condition, age, and susceptibility of the patient.

For peroral application, pharmaceutical compositions can be packaged, for example, into tablets or capsules together with pharmaceutically acceptable additives, such as binding agents (for example, peptized maize starch, polyvinyl pyrrolidinone or hydroxypropyl methylcellulose). Fillers (for example, lactose, microcrystalline cellulose, calcium—hydrophosphate; magnesium stearate, talk or silicon oxide: potato starch or starchy sodium glycolate); or wetting agents (for example, sodium laurylsulfate). Tablets may be coated. Liquid oral compositions can be prepared in the form of, for example, solutions, syrups or suspensions. Such liquid compositions can be obtained by common methods using pharmaceutically acceptable additives, such as suspending agents (for example, cellulose derivatives); emulsifiers (for example, lecithin), diluents (purified vegetable oils); and preservatives (for example, methyl or propyl-n-hydroxybenzoates or sorbic acid). The compositions can also contain appropriate buffering salts, flavoring agents, pigments, and sweeteners.

The contents of the active ingredient in these compositions varies between 0.1 percent and 99.9 percent of the composition weight, preferably, between 5 and 90 percent.

The toxicity of these thrombin inhibitors was measured using standard pharmaceutical procedures on experimental animals to measure LD₅₀ (a lethal dose for 50% of the population). For preferred compounds of this invention, the LD₅₀ dose was in excess of 367 mg/kg, which is consistent with the lethal dose of argothroban after clinical testing, having LD₅₀=475 mg/kg.

For the subject matter of this invention to be more understandable, following below are several examples illustrating the synthesis of new compounds and materials that are intermediate products of their synthesis, accompanied by a description of methods that were used to study the antithrombotic activity of the new compounds claimed as an invention.

The examples are only illustrations, and the idea of this invention is in no way limited to the scope of the examples given below.

### EXAMPLE 1

### SYNTHESIS OF AN INTERMEDIATE PRODUCT OF 3-(3-CHLOROPROPOXY)-5-METHYLPHENOL

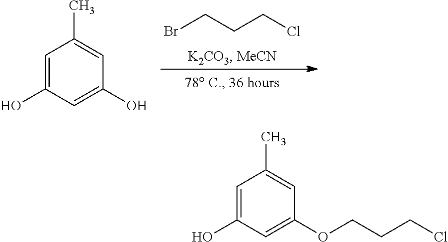

A mixture of 3.8 g (27 mmol) of orcin hydrate, 4.8 g (30 mmol) of 1-bromo-3-chloropropane, and 4.0 g (29 mmol) of potassium carbonate was boiled in 30 ml of acetonitrile at stirring for 36 hours. The reaction mixture was then evaporated, dissolved in 30 ml of an ether, washed twice by 15 ml of a saturated solution of potassium carbonate, the water layer was discarded, and the ether layer was extracted 3 times by 15 ml of a 10% solution of sodium hydroxide. The ether layer was discarded, the water layer was carefully acidified with concentrated HCl, and then extracted with 3 by 15 ml of ester. The ether extracts were joined, washed with small quantities of a saturated solution of sodium hydrocarbonate, and dried with anhydrous sodium sulfate, diluted with/approximately ⅓rd part (by volume) of hexane, and filtered through a layer of silica gel. Evaporation yielded 1.7 g of yellow oil, a mixture of about 70% orcin (Rf 0.10) and about 30% 3-(2-chloropropoxy)-5-methylphenol (Rf 0.26, yield about 1.2 g (22% per pure substance)).

A similar method was used to produce 3-(2-chloroethoxy)-5-methylphenol (Rf 0.26, yield about 1.1 g (20% per pure substance)) from orcin hydrate and 1-bromo-2-chloroethane, and 3-(4-chlorobutoxy)-5-methyl phenol was obtained from orcin hydrate and 1-bromo-4-chlorobutane.

### EXAMPLE 2

### SYNTHESIS OF AN INTERMEDIATE PRODUCT OF 3-(3-CHLOROPROPOXY)-5-METHYLPHENYL ESTER OF BENZENE SULFONIC ACID

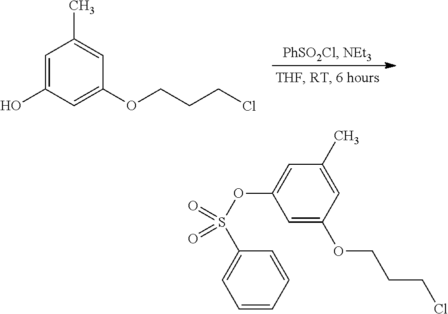

3 g (17 mmol) of benzene sulfochloride and 2 g (20 mmol) of triethylamine were added to a solution of 1.6 g of the mixture of the preceding example in 30 ml of dry tetrahydrofuran (THF). The mixture was stirred for 7 hours, the precipitate of triethylammonium hydrochloride was filtered off and evaporated. The resulting oil was dissolved in 20 ml of an ether and washed several time in 10 ml of 10-12% aqueous solution of ammonia to separate excess unreacted benzene sulfochloride (control by thin-layer chromatography (TLC)) and then 10 ml of approximately 20% hydrochloric acid. Drying with anhydrous sodium sulfate and evaporation gave 1.94 g of yellow oil containing approximately equal quantities of 3-(3-chloropropoxy)-5-methylphenyl ester of benzene sulfonic acid (Rf 0.36) and dibenzoylsulfonic ester of orcin (Rf 0.25) according to TLC.

Similarly, 3-(2-chloroethoxy)-5-methylphenol, 3-(3-chloropropoxy)-5-methylphenol, and 3-(4-chlorobutoxy)-5-methylphenol and appropriate arylsulfochlorides gave:
3-(3-chloropropoxy)-5-methylphenyl ester of 2-chlorobenzene sulfonic acid (77% per pure substance)
3-(3-chloropropoxy)-5-methylphenyl ester of 2-fluorobenzene sulfonic acid (88%).
3-(3-chloropropoxy)-5-methylphenyl ester of 2-carbomethoxy benzene sulfonic acid (56%).
3-(2-chloroethoxy)-5-methylphenyl ester of benzene sulfonic acid (72%).
3-(2-chloroethoxy)-5-methylphenyl ester of 2-chlorobenzene sulfonic acid (35%).
3-(2-chloroethoxy)-5-methylphenyl ester of 2-fluorobenzene sulfonic acid (34%).
3-(2-chloroethoxy)-5-methylphenyl ester of 2-carbomethoxy benzene sulfonic acid (37%).
3-(4-chlorobutoxy)-5-methylphenyl ester of benzene sulfonic acid (45%).
3-(4-chlorobutoxy)-5-methylphenyl ester of 2-chlorobenzene sulfonic acid (27%).
3-(4-chlorobutoxy)-5-methylphenyl ester of 2-fluorobenzene sulfonic acid (32%).
3-(4-chlorobutoxy)-5-methylphenyl ester of 2-carbomethoxy benzene sulfonic acid (21%).

### EXAMPLE 3

### SYNTHESIS OF AN INTERMEDIATE PRODUCT OF 3-(3-IODOPROPOXY)-5-METHYLPHENYL ESTER of 2-CHLOROBENZENE SULFONIC ACID

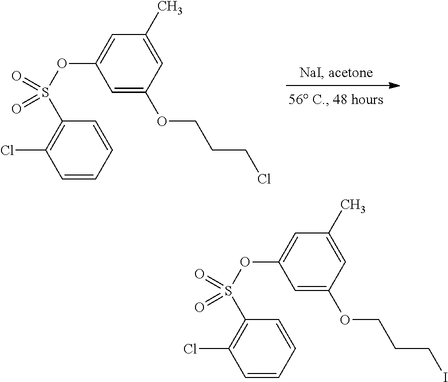

hereinafter, for briefness ClPhO-3-I

2 g (13 mmol) of calcined sodium iodide was added to 2.6 g of a mixture containing 3-(3-chloropropoxy)-5-methylphenyl ester of 2-chlorobenzene sulfonic acid produced similarly to the above example in 30 ml of dry acetone and boiled for 48 hours. The reaction mixture was then diluted with 10 ml of hexane and evaporated. The result was 2.45 g of light-yellow oil containing 3-(2-iodoethoxy)-5-methylphenyl ester of benzene sulfonic acid (Rf 0.35) and a respective dibenzoyl sulfonic ester of orcin (Rf 0.25).

A similar technique was used to process the appropriate chlorides into:
3-(3-iodopropoxy)-5-methylphenyl ester of benzene sulfonic acid
3-(3-iodopropoxy)-5-methylphenyl ester of 2-fluorobenzene sulfonic acid
3-(3-iodopropoxy)-5-methylphenyl ester of 2-carbomethoxy benzene sulfonic acid
3-(2-iodoethoxy)-5-methylphenyl ester of benzene sulfonic acid
3-(2-iodoethoxy)-5-methylphenyl ester of 2-chlorobenzene sulfonic acid
3-(2-iodoethoxy)-5-methylphenyl ester of 2-fluorobenzene sulfonic acid
3-(2-iodoethoxy)-5-methylphenyl ester of 2-carbomethoxy benzene sulfonic acid
3-(4-iodobutoxy)-5-methylphenyl ester of benzene sulfonic acid
3-(4-iodobutoxy)-5-methylphenyl ester of 2-chlorobenzene sulfonic acid
3-(4-iodobutoxy)-5-methylphenyl ester of 2-fluorobenzene sulfonic acid
3-(4-iodobutoxy)-5-methylphenyl ester of 2-carbomethoxy benzene sulfonic acid

### EXAMPLE 4

### SYNTHESIS OF 4-AMINO-1-(3-(3-METHYL-5-(2-CHLOROBENZENE SULFONYLOXY)PHENOXY)PROPYL)-PYRIDINIUM IODIDE (HC_{—}023S_IOC)

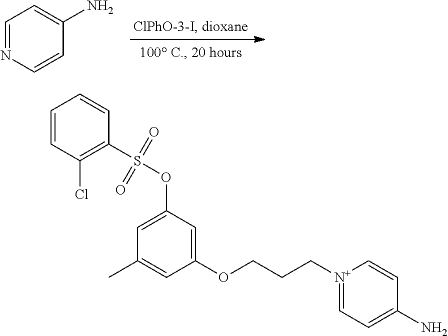

A mixture of 0.55 g of “raw iodide” (from the previous example) (calculated for 70% of active substance) and 0.08 g (0.85 mmol) of 4-aminopyridine in 10 ml of dry dioxane was boiled for 20 hours. After the mixture cooled off, the solution was evaporated, and the resulting oil was ground with a few portions of ether until it turned solid. The solid precipitate was filtered and recrystallized twice from a mixture of dioxane and acetonitrile (5:1), the salt precipitate was filtered off, and washed with ester.

Drying in vacuum gave 0.35 g (65%) of white salt. NMR ¹H (Bruker DRX500, 500 MHz, DMSO-d6, m.d., J Hz): 2.21 s, 3H; 3.91 t, 2H, J=5.49; 2.18 m, 2H, J=6.10; 4.26 t, 2H, J=6.71; 6.40 s, 1H, 6.50 s, 1H, 6.68 s, 1H; 7.59 t, 1H, J=7.94, 7.83 t, 1H, J=7.94, 7.87 d, 1H, J=7.93, 7.95 d, 1H, J=7.93; 6.80 d, 2H, J=6.72, 8.17 d, 2H, J=6.72; 8.07 s, 2H.

A similar technique was used to process appropriate iodides and heterocyclic compounds, thiourea, and thiourea derivatives into:

### 4-amino-1-(3-(3-methyl-5-(benzene sulfonyloxy)phenoxy)propyl)-pyridinium iodide (HC_{—}016s_IOC)

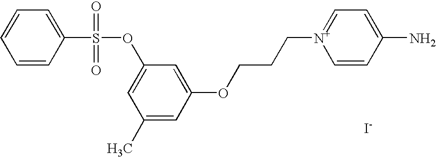

Yield 78%.

NMR ¹H (Bruker DRX500, 500 MHz, DMSO-d6, m.d., J Hz): 2.20 s, 3H; 3.88 t, 2H, J=5.59; 2.16 m, 2H, J=6.11; 4.25 t, 2H, J=6.71; 6.31 s, 1H, 6.44 s, 1H, 6.66 s, 1H; 7.68 t, 2H, J=7.94, 7.82 t, 1H, J=7.94, 7.87 d, 2H, J=7.32; 6.81 d, 2H, J=6.72, 8.17 d, 2H, J=6.72; 8.09 s, 2H

### 2-amino-1-(3-(3-methyl-5-(benzene sulfonyloxy)phenoxy)propyl)-thiazolium iodide (HC_{—}017s_IOC)

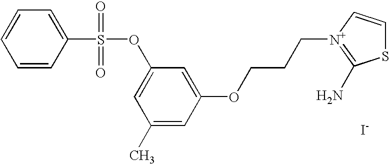

Yield 65%.

NMR ¹H (Bruker DRX500, 500 MHz, DMSO-d6, m.d., J Hz): 2.21 s, 3H; 3.93 t, 2H, J=6.11; 2.11 m, 2H, J=6.10; 4.15 t, 2H, J=6.71; 6.35 s, 1H, 6.44 s, 1H, 6.68 s, 1H; 7.69 t, 2H, J=7.33, 7.84 t, 1H, J=7.32, 7.88 d, 2H, J=7.93; 7.02 d, 1H, J=4.27, 7.42 d, 1H, J=4.27; 9.42 s, 2H

### 3-(3-methyl-5-(benzene sulfonyloxy)phenoxy)propyl-isothiouronium iodide (HC_{—}018s_IOC)

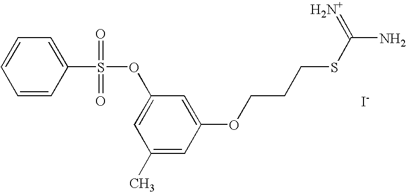

Yield 80%.

NMR ¹H (Bruker DRX500, 500 MHz, DMSO-d6, m.d., J Hz): 2.21 s, 3H; 3.95 t, 2H, J=6.10; 2.00 m, 2H, J=6.71; 3.25 t, 2H, J=7.32; 6.40 s, 1H, 6.25 s, 1H, 6.74 s, 1H; 7.69 t, 2H, J=7.94. 7.84 t, 1H, J=7.93, 7.89 d, 2H, J=7.33; 9.03 s, 4H

### 4-amino-1-(2-(3-methyl-5-(benzene Sulfonyloxy)phenoxy)ethyl)-pyridinium iodide (HC_{—}019s_IOC)

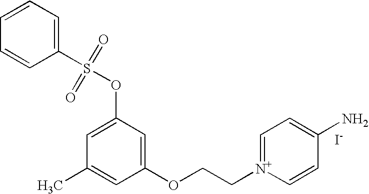

Yield 60%.

NMR ¹H (Bruker DRX500, 500 MHz, DMSO-d6, m.d., J Hz): 2.20 s, 3H; 4.24 t, 2H, J=4.88; 4.48 t, 2H, J=4.89; 6.39 s, 1H, 6.45 s, 1H, 6.73 s, 1H; 7.68 t, 2H, J=7.93, 7.82 t, 1H, J=7.93, 7.87 d, 2H, J=7.32; 6.82 d, 2H, J=7.32, 8.18 d, 2H, J=7.33; 8.14 s, 2H

### 2-(3-methyl-5-(benzene sulfonyloxy)phenoxy)ethyl-isothioronium iodide (HC_{—}020s_IOC)

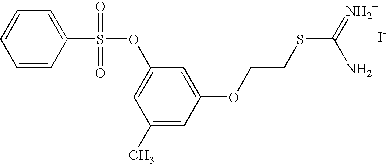

Yield 45%.

NMR ¹H (Bruker DRX500, 500 MHz, DMSO-d6, m.d., J Hz): 2.22 s, 3H; 4.11 t, 2H, J=5.49; 3.54 t, 2H, J=5.49; 6.41 s, 1H, 6.48 s, 1H, 6.76 s, 1H; 7.69 t, 2H, J=7.93, 7.84 t, 1H, J=7.93, 7.89 d, 2H, J=7.32; 9.10 s, 4H

### 2-(3-methyl-5-(2-chlorobenzene sulfonyloxy)phenoxy)ethyl-isothiouronium iodide (HC_{—}024s_IOC)

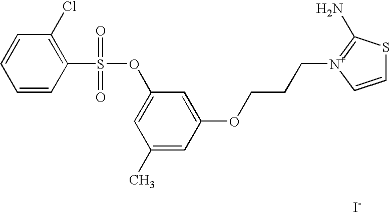

Yield 53%.

NMR ¹H (Bruker DRX500, 500 MHz, DMSO-d6, m.d., J Hz): 2.21 s, 3H; 3.95 t, 2H, J=5.50; 2.12 m, 2H, J=5.50; 4.15 t, 2H, J=6.10; 6.42 t, 1H, 6.51 s, 1H, 6.70 s, 1H; 7.59 t, 1H, J=7.32, 7.83 t, 1H, J=7.94, 7.88 d, 1H, J=7.94, 7.95 d, 1H, J=7.94; 7.01 d, 1H, J=4.27, 7.42 d, 1H, J=4.27; 9.39 s, 2H

### 3-(3-methyl-5-(2-chlorobenzene sulfonyloxy)phenoxy)propyl-isothiouronium iodide (HC_{—}026s_IOC)

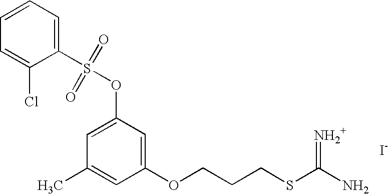

Yield 55%.

NMR ¹H (Bruker DRX500, 500 MHz, DMSO-d6, m.d., J Hz): 2.22 s, 3H; 3.97 t, 2H, J=6.10; 2.01 m, 2H, J=7.33, J=6.10; 4.26 t, 2H, J=7.33; 6.47 s, 1H, 6.51 s, 1H, 6.75 s, 1H; 7.60 t, 1H, J=7.93, 7.84 t, 1H, J=7.94, 7.88 d, 1H, J=7.93, 7.96 d, 1H, J=7.94; 8.95 s, 2H, 9.07 s, 2H

### 4-amino-1-(2-(3-methyl-5-(2-chlorobenzene sulfonyloxy)phenoxy)ethyl)-pyridinium iodide (HC_{—}025s _IOC)

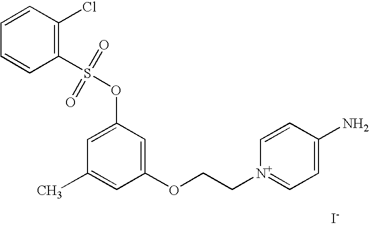

Yield 58%.

NMR ¹H (Bruker DRX500, 500 MHz, DMSO-d6, m.d., J Hz): 2.20 s, 3H; 4.26 t, 2H, J=4.88; 4.49 t, 2H, J=4.88; 6.45 s, 1H, 6.51 s, 1H, 6.74 s, 1H; 7.58 t, 1H, J=7.93, 7.84 t, 1H, J=7.94, 7.88 d, 1H, J=7.93, 7.94 d, 1H, J=7.94; 6.82 d, 2H, J=7.32, 8.18 d, 2H, J=7.33; 8.14 s, 2H.

In a similar way, by techniques described in examples 1-4, compounds were synthesized from various aryl sulfonyl chlorides and heterocyclic sulfonyl chlorides. Chemical formulae, mass-spectrometric parameters, and the computed scoring functions of the synthesized compounds are presented in Table 2. The compounds could be obtained in the form of iodides, bromides, chlorides, or other salts.

### EXAMPLE 5

### SYNTHESIS OF THE COMPOUNDS

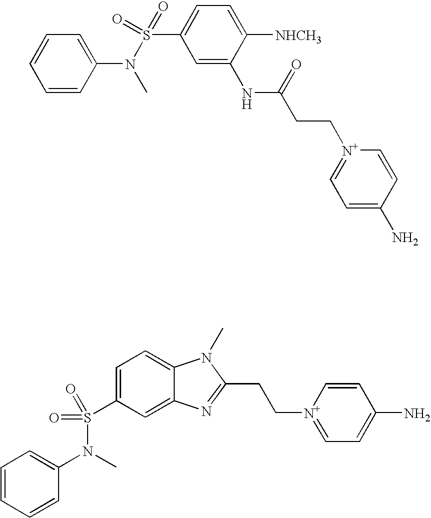

### 1. 4-Chloro-3-nitrobenzene-1-sulfonyl chloride

o-Nitrochloroaniline (15 g) was added into 30 ml of chlorosulfonic acid with stirring and heated at 100° C. for 2 h, followed by 2 h at 110° C. and 5 h at 127° C. The reaction mixture was cooled to room temperature and poured into crushed ice (140 g). The precipitate was filtered; the filter cake was rinsed with ice water and dried in air. The crop was 15 g of 4 chloro-3-nitrobenzene-1 sulfonyl chloride.

### 2. 4- Chloro-N-methyl-3-nitro-N-phenylbenzene sulfonamide

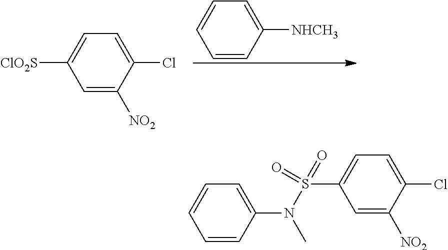

4-Chloro-3-nitrobenzene-1-sulfonyl chloride (10.6 g, 0.041 mol) was dissolved in toluene (50 ml); and triethylamine (4.14 g, 0.041 mol) was then added. To the resulting solution, N-methylaniline (4.4 g, 0.041 mol) was added under stirring. The reaction mixture was incubated at 70-80° C. for 1 h. Thereafter, it was allowed to cool. The cooled solution was washed twice with 30 ml of water and concentrated under vacuum. The residue was recrystallized from ethanol. The yield of 4-chloro-N-methyl-3-nitro-N-phenylbenzene sulfonamide was 9.4 g (61%).

### 3. N-methyl-4-(methylamino)-3-nitro-N-phenylbenzene sulfonamide

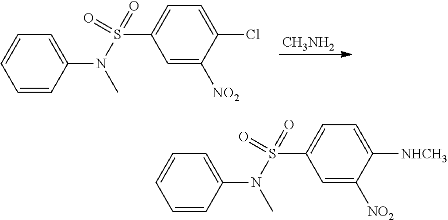

A solution of 4-chloro-N-methyl-3-nitro-N-phenylbenzoyl sulfonamide (9.4 g, 0.029 mol) in ethanol (50 ml) was combined with 25 ml of an aqueous solution of 40% methylamine. The reaction mixture was heated to 70° C. and stirred at this temperature for 1 h. After cooling and filtering, the filter cake was washed with ethanol and dried at 60° C. The yield of N-methyl-4-(methylamino)-3-nitro-N-phenylbenzoyl sulfonamide was 9.0 g (97%).

### 4. 3-amino-N-methyl-4-(methylamino)-N-phenylbenzene sulfonamide

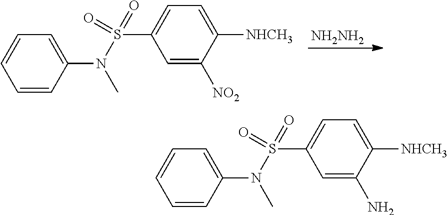

N-Methyl-4-(methylamino)-3-nitro-N-phenylbenzoyl sulfonamide (9 g, 0.028 mol) was dissolved in isopropanol (90 ml). To this solution, hydrazine hydrate (11 ml), activated charcoal (2 g), and FeCl₃ 6H₂O (0.5 g in 10 ml ethanol) were added. The reaction mixture was boiled for 8 h. The charcoal was removed by filtration. The filtrate was evaporated to dryness. The yield of 3-amino-N-methyl-4-(methylamino)-N-phenylbenzene sulfonamide was 8.1 g (99%).

### 5. 3-chloro-N-(5-(N-methyl-N-phenyl sulfamoyl)-2-(methylamino)phenyl)propanamide

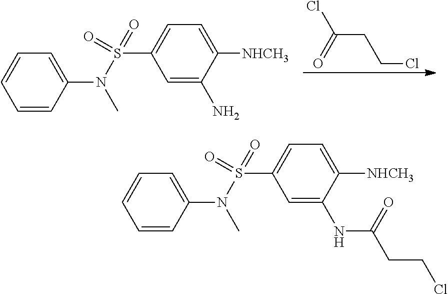

To a solution of 3-amino-N-methyl-4-(methylamino)-N-phenylbenzene sulfonamide (5.4 g, 0.018 mol) and triethylamine (1.81 g, 0.018 mol) in dimethylformamide (16 ml) being cooled on ice (˜5° C.), chloropropionyl chloride (2.32 g, 0.018 mol) was added. The reaction was stirred at room temperature for 5 h. Thereupon, water (14 ml) and acetonitrile (5 ml) were added for 5 h. The precipitate formed was filtered. The yield of 3-chloro-N-(5-(N-methyl-N-phenylsulfamoyl)-2-(methylamino)phenyl)propanamide was 3.1 g (45%).

### 6. 4-amino-1-(3-(5-(N-methyl-N-phenylsulfamoyl)-2-(methylamino)phenylamino)-3-oxopropyl)pyridinium chloride.

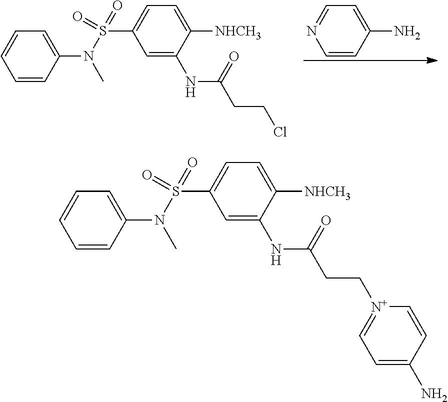

3-Chloro-N-(5-(N-methyl-N-phenylsulfamoyl)-2-(methylamino)phenyl)propanamide (1 g, 0.0026 mol) and 4-aminopyridinium (0.73 g, 0.0078 mol) were boiled in anhydrous acetone (50 ml) for 50 h. The residue was filtered and subjected to crystallization from a 10:1 mixture of acetonitrile with ethanol.

The Yield of 4-amino-1-(3-(5-(N-methyl-N-phenylsulfamoyl)-2-(methylamino)phenylamino)-3-oxopropyl)pyridinium chloride was 0.54 g (43%).

### 7. 4-amino-1-(2-(1-methyl-5-(N-methyl-N-phenylsulfamoyl)-1H-benzo[d]imidazol-2-yl)ethyl)pyridinium chloride.

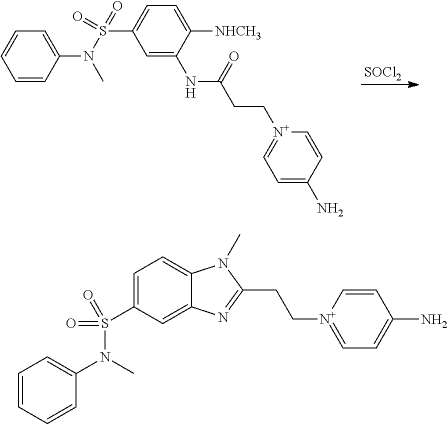

To a suspension of 4-amino-1-(3-(5-(N-methyl-N-phenylsulfamoyl)-2-(methylamino)phenylamino)-3-oxopropyl)pyridinium chloride (0.2 g, 0.00042 mol) in acetonitrile (8 ml), thionyl chloride (0.2 ml) was added. After boiling the reaction mixture for 10 min, it was left to stand at room temperature for 24 h and then diluted with diethyl ether (8 ml). The precipitate formed was collected by filtration and crystallized from a 10:1 mixture of acetonitrile with dehydrated ethanol. The yield of 4-amino-1-(2-(1-methyl-5-(N-methyl-N-phenylsulfamoyl)-1H-benzo[d]imidazol-2-yl)ethyl) pyridinium chloride was 0.055 g (26%).

In a similar way, by techniques described in example 5, various compounds were synthesized, for which chemical formulae, mass-spectrometric parameters, and the computed scoring functions are presented in Table 3. The compounds could be obtained in the form of iodides, bromides, chlorides, or other salts.

### EXAMPLE 6

### STUDY OF THE EFFECT OF TEST COMPOUNDS ON THROMBIN ACTIVITY

The effect of the synthesized substances on thrombin activity was studied by measuring the hydrolysis rate of specific low molecular weight substrates with thrombin in an aqueous buffering solution in the absence and presence of these compounds. One of these substrates was chromogenic substrate Chromozim TH (CTH): N-(p-Tosyl)-Gly-Pro-Arg-pNA [Sonder S A, Fenton J W 2nd. Thrombin Specificity with Tripeptide Chromogenic Substrates: Comparison of Human and Bovine Thrombins with and without Fibrinogen Clotting Activities. Clin. Chem., 1986, 32(6):934-937]. Another substrate that was used in a number of experiments was fluorogenic substrate BOC-Ala-Pro-Arg-AMC (S), wherein BOC is butoxycarbonyl residue, and AMC is 7-amino-4-methylcoumaryl [Kawabata S, Miura T, Morita T, Kato H, Fujikawa K, Ivanaga S, Takada K, Kimura T, Sakakibara S. Highly Sensitive peptide-4-methylcoumaryl-7-amide Substrates for Blood-Clotting Proteases and Trypsin. Eur. J. Biochem., 1988, 172(1):17-25].

The holes of a common 96-hole board were filled with a buffer containing 140 mM of NaCl, 20 mM of HEPES, and 0.1% polyethylene glycol (Mw=6,000), at pH=8.0. A substrate (final concentration in a hole—100 mcM), thrombin (final concentration—190 pM), and the test compound (proposed thrombin inhibitor) at different concentrations (from 0.002 mM to 3.3 mM) were added. When a chromogenic substrate was used, accumulation of the colored reaction product—para-nitroaniline—was followed on a spectrophotometric Molecular Devices board reader (Thermomax, U.S.), measuring the increase in optical density on the 405 nm wavelength. In the case of a fluorogenic substrate, thrombin splits off from it aminomethyl coumaryl that fluoresces significantly in free form during hydrolysis (excitation λ—380 nm and emission λ—440 nm). The reaction kinetics was registered on a fluorometric Titertek Fluoroskan board reader (LabSystem, Finland).

The initial reaction rate was measured as the tangent of the kinetic curve inclination angle on a straight section (first 10 to 15 minutes of registration). Reaction rate without an inhibitor was a ssumed to be 100%. The mean arithmetic value of two independent measurements was used as the end result.

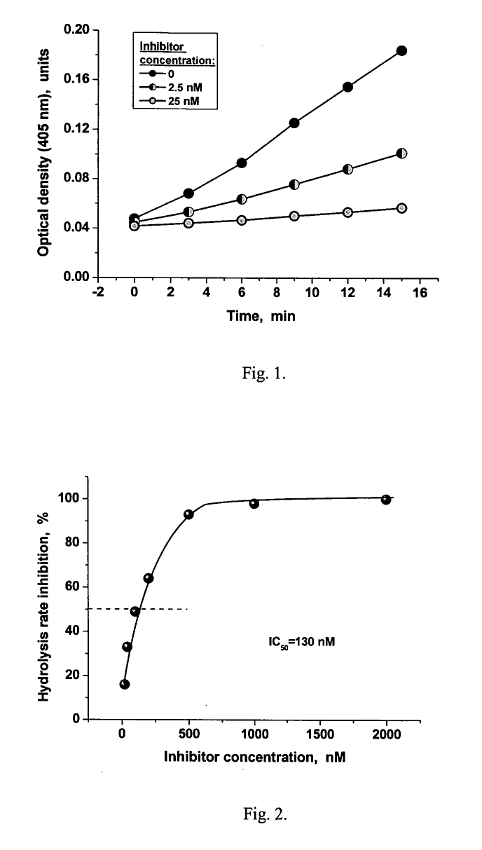

FIG. 1 shows examples of characteristic kinetic hydrolysis curves for chromogenic substrate Chromozim TH (CTH) under the effect of thrombin in the presence of different concentrations of the compound HC-019s-IOC (see: Table 4). The kinetic hydrolysis curve in the absence of an inhibitor was used as control.

FIG. 2 shows the relationship between the extent of CTH hydrolysis inhibition and concentration in the system of another newly synthesized compound (HC-018s-IOC), which is a highly effective thrombin inhibitor (see: Table 4).

Data on the extent of the inhibiting effect of a number of newly synthesized compounds on thrombin activity are given in Table 4.

The results obtained as above show, therefore, that all newly synthesized compounds are direct thrombin inhibitors. The extent of inhibition is different for different compounds, but a majority of new compounds are highly effective thrombin inhibitors, being suitable for use as a base for pharmaceutical compositions used to control thrombin-dependent thromboembolic conditions, and also for use in research.

TABLE 1
List of Key Articles Published on Various Thrombin Inhibitors

| | PDB | | | |
|---|---|---|---|---|
| No. | complex | Inhibitor structure | Article | Comments |
| 1 | 1AD8 | 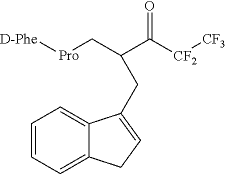 | Malikayil, J. A., Burkhart, J. P., Schreuder, H. A., Broersma Jr., R. J., Tardif, C., Kutcher 3rd, . 3., Mehdi, S., Schatzman, G. L., Neises, B., Peet, N. P.: Molecular design and characterization of an alpha-thrombin inhibitor containing a novel P1 moiety. Biochemistry 36 pp. 1034 (1997) | Covalent inhibitor |
| 2 | 1AWF | Complex natural steroid | Weir, M. P., Bethell, S. S., Cleasby, A., Campbell, C. J., Dennis, R. J., Dix, C. J., Finch, H., Jhoti, H., Mooney, C. J., Patel, S., Tang, C. M., Ward, M., Wonacott, A. J., Wharton, C.W.: Novel natural product 5,5-trans-lactone inhibitors of human alpha- thrombin: mechanism of action and structural studies. Biochemistry 37 pp. 6645 (1998) | Covalent inhibitor |
| 3 | 1AY6 | Macrocyclic peptide | Maryanoff. B. E., Qiu, X., Padmanabhan, K. P., Tulinsky, A., Almond Jr., H. R, Andrade-Gordon. P., Greco, M. N., Kauffman, J. A., Nicolaou, K. C., Liu, A., et al.: Molecular basis for the inhibition of human alpha- thrombin by the macrocyclic peptide cyclotheonamide A. Proc Natl Acad Sci USA 90 pp. 8048 (1993) | |
| 4 | 1BA8 | 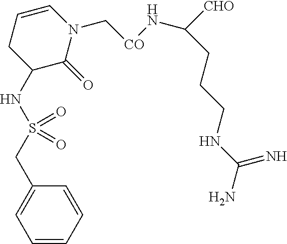 | Krishnan, R., Zhang, E., Hakansson, K., Ami, R. K., Tulinsky, A., Lim-Wilby, M. S., Levy, O. E., Semple. J. E., Brunck, T. K.: Highly selective mechanism-based thrombin inhibitors: structures of thrombin and trypsin inhibited with rigid peptidyl aldehydes. Biochemistry 37 pp. 12094 (1998) | Covalent inhibitor |
| 5 | 1BHX | 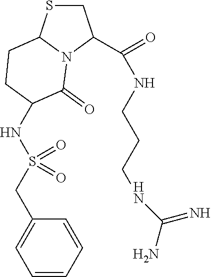 | Wagner, J., Kallen, J., Ehrhardt, C., Evenou, J. P., Wagner, D.: Rational design, synthesis, and X-ray structure of selective noncovalent thrombin inhibitors. J Med Chem 41 pp. 3664 (1998) | |
| 6 | 1D6W | 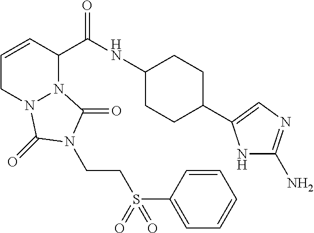 | Krishnan, R., Mochalkin, I., Arni, R., Tulinsky, A.: Structure of Thrombin Complexed with Selective Non-Electrophilic Inhibitors Having Cyclohexyl Moieties at P1 Acta Crystallogr., Sect. D 56 pp. 294 (2000) | |
| 7 | 1DWB | 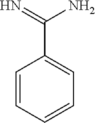 | Banner, D. W., Hadvary, P.: Crystallographic analysis at 3.0-A resolution of the binding to human thrombin of four active site-directed inhibitors. J Biol Chem 266 pp. 20085 (1991) | K_{d} = 343 mcM |
| 8 | 1DWC | 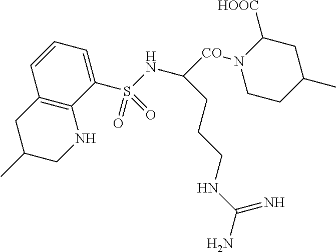 | Banner, D. W., Hadvary, P.: Crystallographic analysis at 3.0-A resolution of the binding to human thrombin of four active site-directed inhibitors. J Biol Chem 266 pp. 20085 (1991) | MD-805 (MITSUBISHI INHIBITOR) |
| 9 | 1DWD | 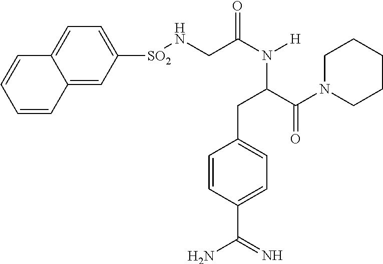 | Banner, D. W., Hadvary, P.: Crystallographic analysis at 3.0-A resolution of the binding to human thrombin of four active site-directed inhibitors. J Biol Chem 266 pp. 20085 (1991) | N═ALPHA═(2- NAPHTHYL- SULFONYL- GLYCYL)- PARA- AMINO- ALANYL- PIPERIDINE |
| 10 | 1DWE | 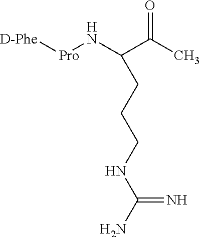 | Banner, D. W., Hadvary, P.: Crystallographic analysis at 3.0-A resolution of the binding to human thrombin of four active site-directed inhibitors. J Biol Chem 266 pp. 20085 (1991) | |
| 11 | 1EOJ | Peptide inhibitor | Slon-Usakiewicz, J. J., Sivaraman. J., Li, Y., Cygler. M, Konishi, Y.: Design of P1’ and P3’ Residues of Trivalent Thrombin Inhibitors and Their Crystal Structures Biochemistry 39 pp. 2384 (2000) | |
| 12 | 1EOL | Peptide inhibitor | Slon-Usakiewicz, J. J., Sivaraman, J., Li, Y., Cygler, M., Konishi, Y.: Design of P1′ and P3′ Residues of Trivalent Thrombin Inhibitors and Their Crystal Structures Biochemistry 39 pp. 2384 (2000) | |
| 13 | 1G30 | 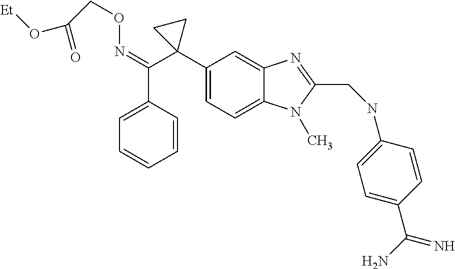 | Nar, H., Bauer, M., Schmid, A., Stassen, J., Wienen, W., Priepke, H. W., Kauffmann, I. K., Ries, U. J., Hauel, N. H.: Structural Basis for Inhibition Promiscuity of Dual Specific Thrombin and Factor Xa Blood Coagulation Inhibitors Structure 9 pp. 29 (2001) | |
| 14 | 1G32 | 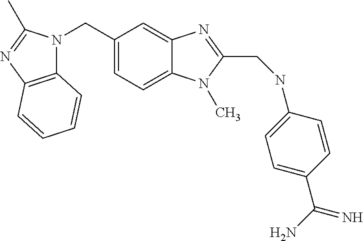 | Nar, H., Bauer, M., Schmid, A., Stassen, J., Wienen, W., Priepke, H. W., Kauffmann, I. K., Ries, U. J., Hauel, N. H.: Structural Basis for Inhibition Promiscuity of Dual Specific Thrombin and Factor Xa Blood Coagulation Inhibitors Structure 9 pp. 29 (2001) | |
| 15 | 1G37 | 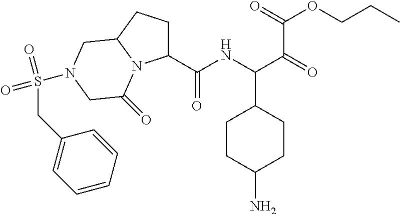 | Bachand, B., Tarazi, M., St- Denis, Y., Edmunds, J. J., Winocour, P. D., Leblond, L., Siddiqui, M. A.: Potent and Selective Bicyclic Lactam Inhibitors of Thrombin. Part 4: Transition State Inhibitors Bioorg. Med. Chem. Lett. 11 pp. 287 (2001) | |
| 16 | 1H8D 1H8I | 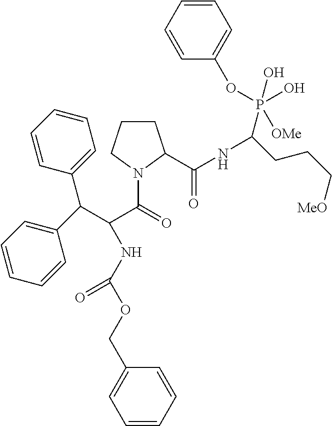 | Skordalakes, E., Dodson, G. G, Green, D. S., Goodwin, C. A., Scully, M. F., Hudson, H. R, Kakkar, V. V., Deadman, J. J.: Inhibition of Human Alpha-Thrombin by a Phosphonate Tripeptide Proceeds Via a Metastable Pentacoordinated Phosphorus Intermediate J. Mol. Biol. 311 pp. 549 (2001) | |
| 17 | 1K21 | 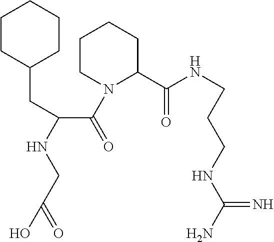 | Dullweber, F., Stubbs, M. T., Musil, D., Sturzebecher, J., Klebe, G.: Factorising Ligand Affinity: A Combined Thermodynamic and Crystallographic Study of Trypsin and Thrombin Inhibition J. Mol. Biol. 313 pp. 593 (2001) | |
| 18 | 1K22 | 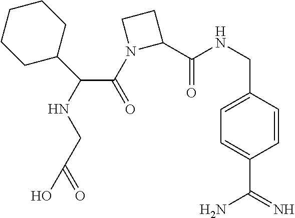 | Dullweber, F., Stubbs, M. T., Musil, D., Sturzebecher, J., Klebe, G.: Factorising Ligand Affinity: A Combined Thermodynamic and Crystallographic Study of Trypsin and Thrombin Inhibition J. Mol. Biol. 313 pp. 593 (2001) | |
| 19 | 1KTS | 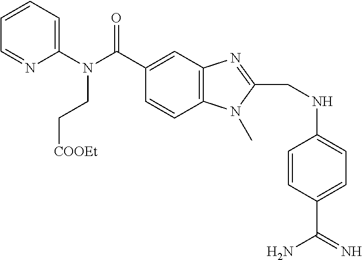 | Hauel, N. H., Nar, H., Priepke, H., Ries, U., Stassen, J. -M., Wienen, W.: Structure- Based Design of Novel Potent Nonpeptide Thrombin Inhibitors J. Med. Chem. 45 pp. 1757 (2002) | |
| 20 | 1KTT | 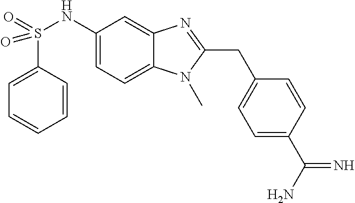 | Hauel, N. H., Nar, H., Priepke, H., Ries, U., Stassen, J. -M., Wienen, W.: Structure- Based Design of Novel Potent Nonpeptide Thrombin Inhibitors J. Med. Chem. 45 pp. 1757 (2002) | |
| 21 | 1NM6 | Complex macrocycle | Nantermet, P. G., Barrow, J. C., Newton, C. L., Pellicore, J. M., Young, M., Lewis, S. D., Lucas, B. J., Krueger, J. A, Mcmasters, D. R., Yan, Y., Kuo, L. C.,Vacca, J. P., Selnick, H. G.: Design and Synthesis of Potent and Selective Macrocyclic Thrombin Inhibitors Bioorg. Med. Chem. Lett. 13 pp. 2781 (2003) | |
| 22 | 1NT1 | Complex macrocycle | Nantermet, P. G., Barrow, J. C., Newton, C. L., Pellicore, J. M, Young, M., Lewis, S. D., Lucas, B. J., Krueger, J. A., Mcmasters, D. R., Yan, Y., Kuo, L. C., Vacca, J. P., Selnick, H. G.: Design and Synthesis of Potent and Selective Macrocyclic Thrombin Inhibitors Bioorg. Med. Chem. Lett. 13 pp. 2781 (2003) | |
| 23 | 1NZQ | 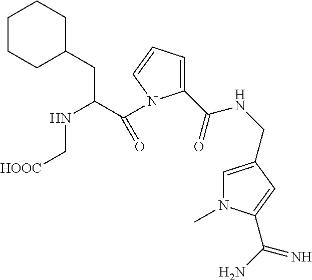 | Lange, U. E., Bauke, D., Hornberger, W., Mack, H., Seitz, W., Hoeffken, H. W.: D- Phe-Pro-Arg Type Thrombin Inhibitors: Unexpected Selectivity by Modification of the P1 Moiety Bioorg. Med. Chem. Lett. 13 pp. 2029 (2003) | |
| 24 | 1QBV | 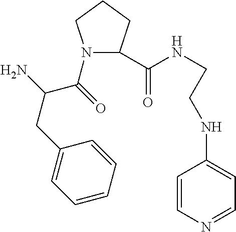 | Bone, R., Lu, T., Illig, C. R., Soll, R. M., Spurlino, J. C.: Structural Analysis of Thrombin Complexed with Potent inhibitors Incorporating a Phenyl Group as a Peptide Mimetic and Aminopyridines as Guanidine Substitutes. J. Med. Chem. 41 pp. 2068 (1998) | |
| 25 | 1RIW | 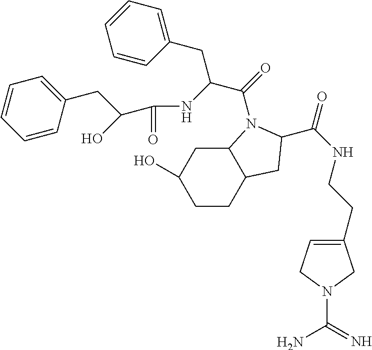 | Hanessian, S., Tremblay, M., Petersen, J. F. W.: The N- Acyloxyiminium Ion Aza- Prins Route to Octahydroindoles: Total Synthesis and Structural Confirmation of the Antithrombotic Marine Natural Product Oscillarin J. Am. Chem. Soc. 126 pp. 6064 (2004) | Oscillarin (natural product) |
| 26 | 1SL3 | 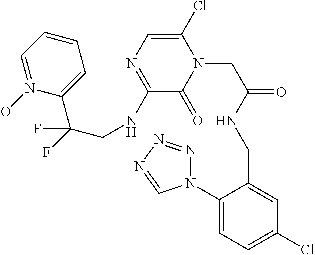 | Young, M. B., Barrow, J. C., Glass, K. L., Lundell, G. F., Newton, C. L., Pellicore, J. M., Rittle, K. E., Selnick, H. G., Stauffer, K. J., Vacca, J. P., Williams, P. D., Bohn, D., Clayton, F. C., Cook, J. J., Krueger, J. A., Kuo, L. C., Lewis, S. D., Lucas, B. J., Mcmasters, D. R., Miller- Stein, C., Pietrak, B. L.: Discovery and Evaluation of Potent P1 Aryl Heterocycle- Based Thrombin Inhibitors J. Med. Chem. 47 pp. 2995 (2004) | |
| 27 | 1T4U | 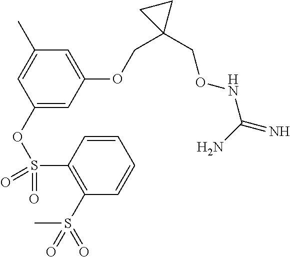 | Lu, T., Markotan, T., Coppo, F., Tomczuk, B., Crysler, C., Eisennagel, S., Spurlino, J., Gremminger, L., Soll, R. M., Giardino, E. C., Bone, R.: Oxyguanidines. Part 2: Discovery of a Novel Orally Active Thrombin Inhibitor Through Structure-Bascd Drug Design and Parallel Synthesis Bioorg. Med. Chem. Lett. 14 pp. 3727 (2004) | |
| 28 | 1T4V | 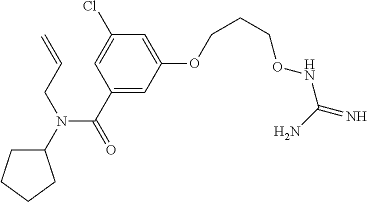 | Lu, T., Markotan, T., Coppo, F., Tomczuk, B., Crysler, C., Eisennagel, S., Spurlino, J., Gremminger, L., Soll, R. M., Giardino, E. C., Bone, R.: Oxyguanidines. Part 2: Discovery of a Novel Orally Active Thrombin Inhibitor Through Structure-Based Drug Design and Parallel Synthesis Bioorg. Med. Chem. Lett. 14 pp. 3727 (2004) | |
| 29 | 1TA2 | 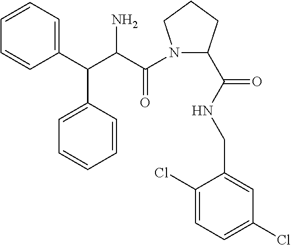 | Tucker, T. J., Brady, S. F., Lumma, W. C., Lewis, S. D., Gardel, S. J., Naylor-Olsen, A. M., Yan, Y., Sisko, J. T., Stauffer, K. J., Lucas, B. Y., Lynch, J. J., Cook, J. J., Stranieri, M. T., Holahan, M. A., Lyle, E. A., Baskin, E. P., Chen, I. -W., Dancheck, K. B., Krueger, J. A., Cooper, C. M., Vacca, J. P.: Design and Synthesis of a Series of Potent and Orally Bioavailable Noncovalent Thrombin Inhibitors that Utilize Nonbasic Groups in the P1 Position J. Med. Chem. 41 pp. 3210 (1998) | |
| 30 | 1TA6 | 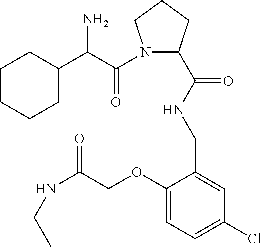 | Tucker, T. J., Brady, S. F., Lumma, W. C., Lewis, S. D., Gardel, S. J., Naylor-Olsen, A. M., Yan, Y., Sisko, J. T., Stauffer, K. J., Lucas, B. Y., Lynch, J. J., Cook, J. J., Stranieri, M. T., Holahan, M. A., Lyle, E. A., Baskin, E. P., Chen, I. -W., Dancheck, K. B., Krueger, J. A., Cooper, C. M., Vacca, J. P.: Design and Synthesis of a Series of Potent and Orally Bioavailable Noncovalent Thrombin Inhibitors that Utilize Nonbasic Groups in the P1 Position J. Med. Chem. 41 pp. 3210 (1998) | |
| 31 | 1TBQ | Peptide inhibitor | van de Locht, A., Lamba, D., Bauer, M., Huber, R., Friedrich, T., Kroger, B., Hoffken, W., Bode, W.: Two heads are better than one: crystal structure of the insect derived double domain Kazal inhibitor rhodniin in complex with thrombin. EMBO J 14 pp. 5149 (1995) | |
| 32 | 1TBR | Peptide inhibitor | van de Locht, A., Lamba, D., Bauer, M., Huber, R., Friedrich, T., Kroger, B., Hoffken, W., Bode, W.: Two heads are better than one: crystal structure of the insect derived double domain Kazal inhibitor rhodniin in complex with thrombin. EMBO J 14 pp. 5149 (1995) | |
| 33 | 1TOM | 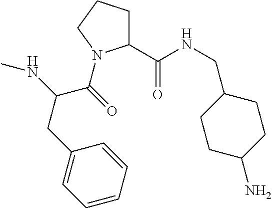 | Lyle, T. A., Chen, Z. G., Appleby, S. D., Freidinger, R. M., Gardell, S. J., Lewis, S. D., Li, Y., Lyle, E. A., Lynch, J. J., Mulichak, A. M., Ng, A. S., NaylorOlsen, A. M., Sanders, W. M.: Synthesis, evaluation, and crystallographic analysis of L-371, 912: A potent and selective active-site thrombin inhibitor. BIOORGANIC & MEDICINAL CHEMISTRY LETTERS 7 pp. 67 (1997) | |
| 34 | 1VZQ | 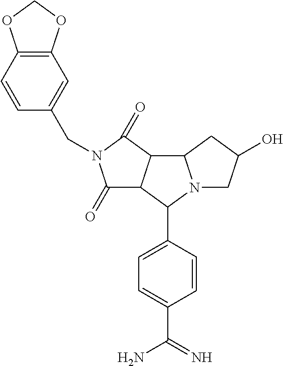 | Schaerer, K., Morgenthaler, M, Seiler, P., Diederich, F., Banner, D. W., Tschopp, T., Obst-Sander, U.: Enantiomerically Pure Thrombin Inhibitors for Exploring the Molecular- Recognition Features of the Oxyanion Hole Helv. Chim. Acta 87 pp. 2517 (2004) | |
| 35 | 1WAY | 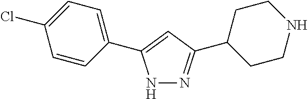 | Hartshorn, M. J., Murray, C. W.; Cleasby, A., Frederickson, M., Tickle, I. J., Jhoti, H.: Fragment-Based Lead Discovery Using X-Ray Crystallography J. Med. Chem. 48 pp. 403 (2005) | Affinity- 400 mcM |
| 36 | 1WBG | 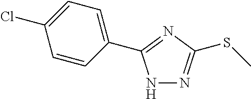 | Hartshom, M. J., Murray, C. W., Cleasby, A., Frederickson, M., Tickle, I. J., Jhoti, H.: Fragment-Based Lead Discovery Using X-Ray Crystallography J. Med. Chem. 48 pp. 403 (2005) | Affinity- 1 mM |
| 37 | 1Z71 | 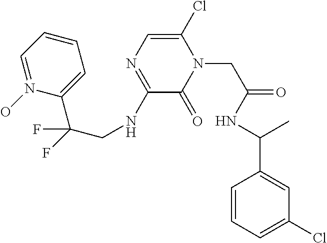 | Nantermet, P. G., Burgey, C. S., Robinson, K. A., Pellicore, J. M, Newton, C. L., Deng, J. Z., Selnick, H. G., Lewis, S. D, Lucas, B. J., Krueger, J. A, Miller-Stein, C., White, R. B., Wong, B., Mcmasters, D. R., Wallace, A. A., Lynch Jr.. J. J, Yan, Y., Chen, Z., Kuo, L., Gardell, S. J., Shafer, J. A., Vacca, J. P., Lyle, T. A.: P(2) Pyridine N-Oxide Thrombin Inhibitors: A Novel Peptidomimetic Scaffold Bioorg. Med. Chem. Lett. 15 pp. 2771 (2005) | |
| 38 | 1ZGI | 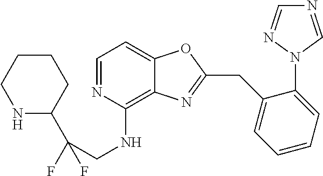 | Deng, J. Z., Mcmasters, D. R., Rabbat, P. M., Williams, P. D., Coburn, C. A., Yan, Y., Kuo, L. C., Lewis, S. D., Lucas, B. J., Krueger, J. A., Strulovici, B., Vacca, J. P., Lyle, T. A., Burgey, C. S.: Development of an Oxazolopyridine Series of Dual Thrombin/Factor Xa Inhibitors Via Structure- Guided Lead Optimization. Bioorg. Med. Chem. Lett. 15 pp. 4411 (2005) | |
| 39 | 1ZGV | 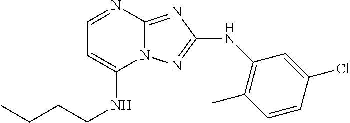 | Deng, J. Z., Mcmasters, D. R., Rabbat, P. M., Williams, P. D., Coburn, C. A., Yan, Y., Kuo, L. C., Lewis, S. D., Lucas, B. J., Krueger, J. A., Strulovici, B., Vacca, J. P., Lyle, T. A., Burgey, C. S.: Development of an Oxazolopyridine Series of Dual Thrombin/Factor Xa Inhibitors Via Structure- Guided Lead Optimization. Bioorg. Med. Chem. Lett. 15 pp. 4411 (2005) | |
| 40 | 1ZRB | 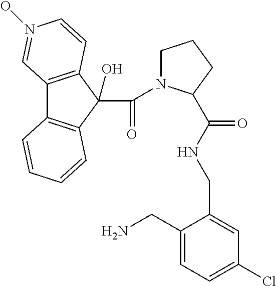 | Stauffer, K. J., Williams, P. D, Selnick, H. G., Nantermet, P. G., Newton, C. L., Homnick, C. F., Zrada, M. M., Lewis, S. D., Lucas, B. J., Krueger, J. A., Pietrak, B. L., Lyle, E. A., Singh, R., Miller- Stein, C., White, R. B., Wong, B., Wallace, A. A., Sitko, G. R., Cook, J. J., Holahan, M. A., Stranieri- Michener, M., Leon, Y. M.: 9-Hydroxyazafluorenes and Their Use in Thrombin Inhibitors J. Med. Chem. 48 pp. 2282 (2005) | |
| 41 | 7KME | 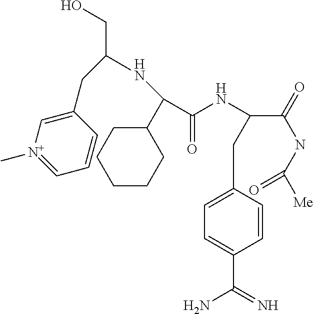 | Mochalkin, I., Tulinsky, A.: Structures of thrombin retro-inhibited with SEL2711 and SEL2770 as they relate to factor Xa binding. Acta Crysrallogr D Biol Crystallogr 55 pp. 785 (1999) | |
| 42 | 8KME | 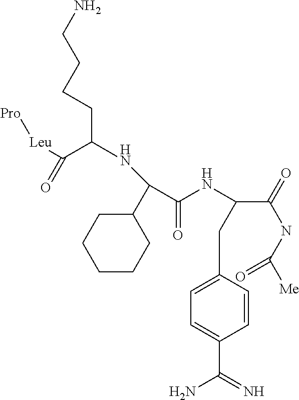 | Mochalkin, I., Tulinsky, A.: Crystal Structures of Thrombin Retror-Inhibited with Sel2711 and Sel2770 as They Relate to Factor Xa Binding To be Published | |
| 43 | 1A2C | Aeruginosin 298A - peptide | Steiner, J. L. R., Murakami, M., Tulinsky, A.: Structure of thrombin inhibited by aeruginosin 298-A from a blue-green alga. J Am Chem Soc 120 pp. 597 (1998) | |
| 44 | 1A3B | 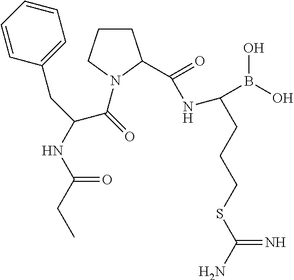 | Zdanov, A., Wu, S., DiMaio, J., Konishi, Y., Li, Y., Wu, X., Edwards, B. F., Martin, P. D., Cygler, M.: Crystal structure of the complex of human alpha-thrombin and nonhydrolyzable bifunctional inhibitors, hirutonin-2 and hirutonin-6. Proteins 17 pp. 252 (1993) | Borolog 1 |
| 45 | 1A3E | 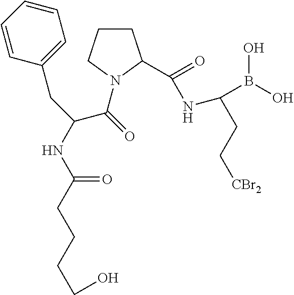 | Zdanov, A., Wu, S., DiMaio, J., Konishi, Y., Li, Y., Wu, X., Edwards, B. F., Martin, P. D., Cygler, M.: Crystal structure of the complex of human alpha-thrombin and nonhydrolyzable bifunctional inhibitors, hirutonin-2 and hirutonin-6. Proteins 17 pp. 252 (1993) | Borolog 2 |
| 46 | 1A46 | 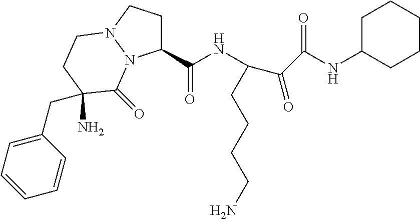 | St Charles, R., Matthews, J. H., Zhang, E. L., Tulinsky, A: Bound structures of novel P3-P1′ beta-strand mimetic inhibitors of thrombin. J Med Chem 42 pp. 1376 (1999) | |
| 47 | 1A4W | 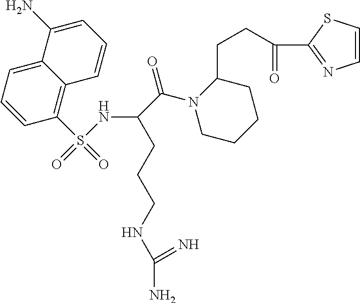 | Matthews, J. H., Krishnan, R., Costanzo, M. J., Maryanoff, B.E, Tulinsky, A.: Crystal structures of thrombin with thiazole-containing inhibitors: probes of the S1′ binding site. Biophys J 71 pp. 2830 (1996) | |
| 48 | 1A5G | 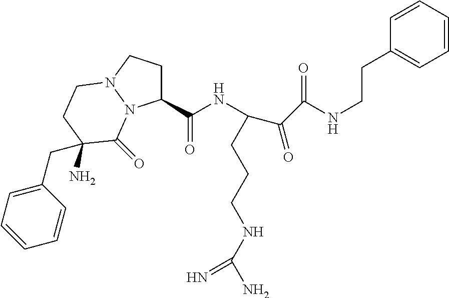 | St Charles, R., Matthews, J. H., Zhang, E. L., Tulinsky, A: Bound structures of novel P3-P1′ beta-strand mimetic inhibitors of thrombin. J Med Chem 42 pp. 1376 (1999) | |
| 49 | 1A61 | 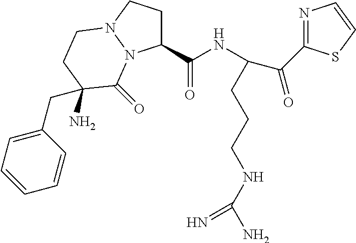 | St Charles, R., Matthews, J. H., Zhang, E. L., Tulinsky, A: Bound structures of novel P3-P1′ beta-strand mimetic inhibitors of thrombin. J Med Chem 42 pp. 1376 (1999) | |
| 50 | 1ABI | D-Phe-Pro- Homoarginine- glycine- hirudin bridge | Qiu, X., Padmanabhan, K. P., Carperos, V. E., Tulinsky, A., Kline, T., Maraganore, J. M., Fenton 2d, . 2.: Structure of the hirulog 3-thrombin complex and nature of the S′ subsites of substrates and inhibitors. Biochemistry 31 pp. 11689 (1992) | |
| 51 | 1ABJ | D-Phe-Pro-arginine | Qiu, X., Padmanabhan, K. P., Carperos, V. E., Tulinsky, A., Kline, T., Maraganore, J. M., Fenton 2d, . 2.: Structure of the hirulog 3-thrombin complex and nature of the S′ subsites of substrates and inhibitors. Biochemistry 31 pp. 11689 (1992) | |
| 52 | 1AE8 | 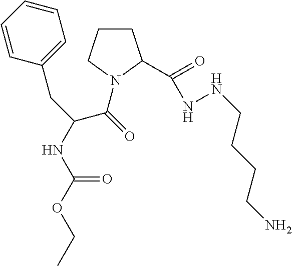 | De Simone, G., Balliano, G., Milla, P., Gallina, C., Giordano, C., Tarricone, C., Rizzi, M., Bolognesi, M., Ascenzi, P.: Human alpha- thrombin inhibition by the highly selective compounds N-ethoxycarbonyl-D-Phe- Pro-alpha-azaLys p- nitrophenyl ester and N- carbobenzoxy-Pro-alpha- azaLys p-nitrophenyl ester: a kinetic, thermodynamic and X-ray crystallographic study. J Mol Biol 269 pp. 558 (1997) | |
| 53 | 1AFE | 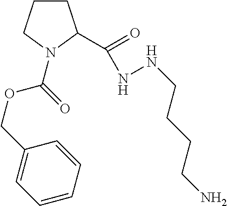 | De Simone, G., Balliano, G., Milla, P., Gallina, C., Giordano, C., Tarricone, C., Rizzi, M., Bolognesi, M., Ascenzi, P.: Human alpha- thrombin inhibition by the highly selective compounds N-ethoxycarbonyl-D-Phe- Pro-alpha-azaLys p- nitrophenyl ester and N- carbobenzoxy-Pro-alpha- azaLys p-nitrophenyl ester: a kinetic, thermodynamic and X-ray crystallographic study. J Mol Biol 269 pp. 558 (1997) | |
| 54 | 1AHT | 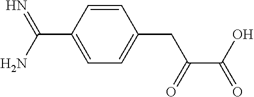 | Chen, Z., Li, Y., Mulichak, A. M., Lewis, S. D., Shafer, J. A.: Crystal structure of human alpha-thrombin complexed with hirugen and p-amidinophenylpyruvate at 1.6 A resolution. Arch Biochem Biophys 322 pp. 198 (1995) | Covalent inhibitor |
| 55 | 1AI8 | 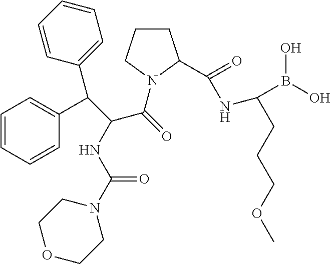 | Bode, W., Turk, D., Karshikov, A.: The refined 1.9-A X-ray crystal structure of D-Phe-Pro-Arg chloromethylketone-inhibited human alpha-thrombin: structure analysis, overall structure, electrostatic properties, detailed active-site geometry, and structure- function relationships. Protein Sci 1 pp. 426 (1992) | |
| 56 | 1AIX | 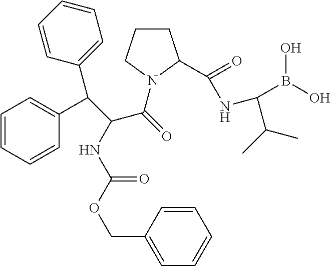 | Bode, W., Turk, D., Karshikov, A.: The refined 1.9-A X-ray crystal structure of D-Phe-Pro-Arg chloromethylketone-inhibited human alpha-thrombin: structure analysis, overall structure, electrostatic properties, detailed active-site geometry, and structure- function relationships. Protein Sci 1 pp. 426 (1992) | |
| 57 | 1B5G | 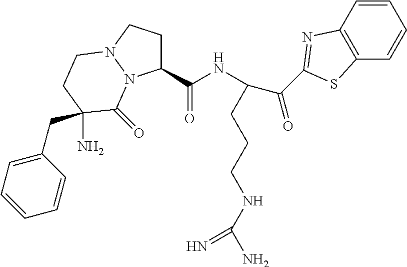 | Charles, R. St., Matthews, J. H., Zhang, E., Tulinsky, A.: Bound structures of novel P3- P1′ beta-strand mimetic inhibitors of thrombin. J Med Chem 42 pp. 1376 (1999) | |
| 58 | 1BB0 | 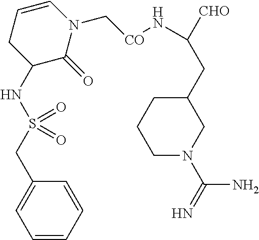 | Krishnan, R., Zhang, E., Hakansson, K., Arni, R. K., Tulinsky, A., Lim-Wilby, M. S., Levy, O. E., Semple, J. E., Brunck, T. K.: Highly selective mechanism-based thrombin inhibitors: structures of thrombin and trypsin inhibited with rigid peptidyl aldehydes. Biochemistry 37 pp. 12094 (1998) | |
| 59 | 1BCU | 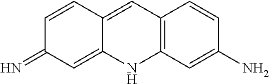 | Conti, E., Rivetti, C., Wonacott, A., Brick, P.: X- ray and spectrophotometric studies of the binding of proflavin to the S1 specificity pocket of human alpha- thrombin. FEBS Lett 425 pp. 229 (1998) | Proflavin. Micromolecular affinity K_{d} ~ 10 mcM |
| 60 | 1BMM | 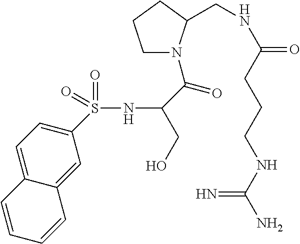 | Malley, M. F., Tabernero, L., Chang, C. Y., Ohringer, S. L., Roberts, D. G., Das, J., Sack, J. S.: Crystallographic determination of the structures of human alpha-thrombin complexed with BMS- 186282 and BMS- 189090. Protein Sci 5 pp. 221 (1996) | |
| 61 | 1BMN | 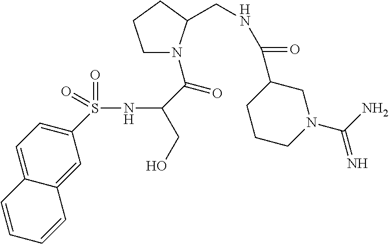 | Malley, M. F., Tabernero, L., Chang, C.Y., Ohringer, S. L., Roberts, D. G., Das, J., Sack, J. S.: Crystallographic determination of the structures of human alpha-thrombin complexed with BMS- 186282 and BMS- 189090. Protein Sci 5 pp. 221 (1996) | |
| 62 | 1C1U | 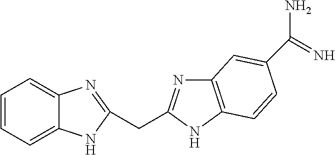 | Katz, B. A, Clark, J. M., Finer-Moore, J. S., Jenkins, T. E., Johnson, C. R., Ross, M. J., Luong, C., Moore, W. R., Stroud, R. M.: Design of Potent Selective Zinc- Mediated Serine Protease Inhibitors Nature 391 pp. 608 (1998) | Zinc present in the active site mediated by a ligand. It is a co- inhibitor |
| 63 | 1C1V | 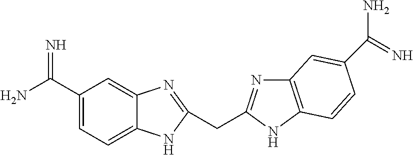 | Katz, B. A., Clark, J. M., Finer-Moore, J. S., Jenkins, T. E., Johnson, C. R., Ross M. J., Luong, C., Moore, W. R., Stroud, R. M.: Design of Potent Selective Zinc- Mediated Serine Protease Inhibitors Nature 391 pp. 608 (1998) | Zinc present in the active site mediated by a ligand. It is a co- inhibitor |
| 64 | 1C1W | 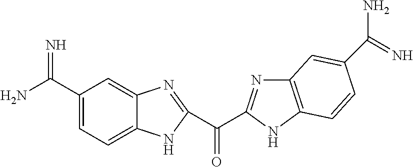 | Katz, B. A., Clark, J. M., Finer-Moore, J. S., Jenkins, T. E., Johnson, C. R., Ross M. J., Luong, C., Moore, W. B., Stroud, R. M.: Design of Potent Selective Zinc- Mediated Serine Protease Inhibitors Nature 391 pp. 608 (1998) | Zinc present in the active site mediated by a ligand. It is a co- inhibitor |
| 65 | 1C4U | 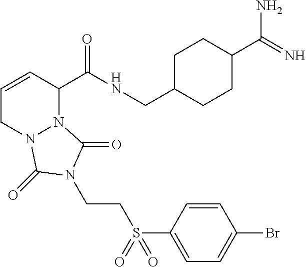 | Krishnan, R., Mochalkin, I., Arni, R. K., Tulinsky, A.: Structure of Thrombin Complexed with Selective Non-Electrophilic Inhibitors Having Cyclohexyl Moieties at P1 Acta Crystallogr., Sect. D 56 pp. 294 (2000) | |
| 66 | 1C4V | 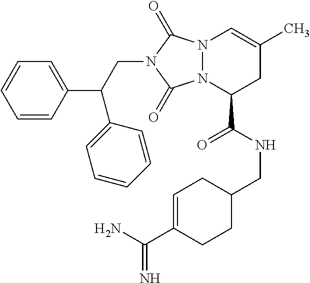 | Krishnan, R., Mochalkin, I., Arni, R. K., Tulinsky, A.: Structure of Thrombin Complexed with Selective Non-Electrophilic Inhibitors Having Cyclohexyl Moieties at P1 Acta Crystallogr., Sect. D 56 pp. 294 (2000) | |
| 67 | 1C4Y | 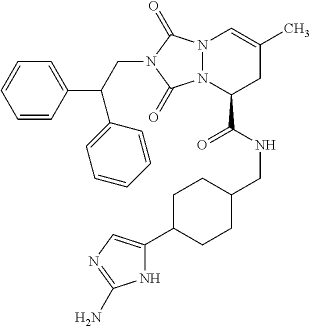 | Krishnan, R., Mochalkin, I., Arni, R. K., Tulinsky, A.: Structure of Thrombin Complexed with Selective Non-Electrophilic Inhibitors Having Cyclohexyl Moieties at P1 Acta Crystallogr., Sect. D 56 pp. 294 (2000) | |
| 68 | 1C5N | 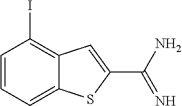 | Katz, B. A., Mackman, R., Luong, C., Radika, K., Martelli, A., Sprengeler, P. A., Wang, J., Chan, H., Wong, L.: Structural Basis for Selectivity of a Small Molecule, S1-Binding, Submicromolar Inhibitor of Urokinase-Type Plasminogen Activator Chem. Biol. 7 pp. 299 (2000) | Human |
| 69 | 1C5O | 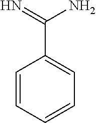 | Katz, B. A., Mackman, R., Luong, C., Radika, K., Martelli, A., Sprengeler, P. A., Wang, J., Chan, H., Wong, L.: Structural Basis for Selectivity of a Small Molecule, S1-Binding, Submicromolar Inhibitor of Urokinase-Type Plasminogen Activator Chem. Biol. 7 pp. 299 (2000) | Human |
| 70 | 1W7G | 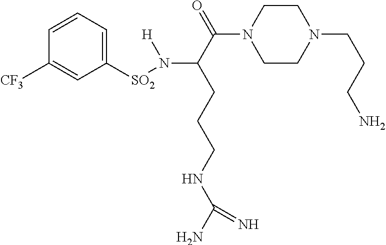 | Salvagnini, C., Michaux, C., Remiche, J., Wouters, J., Charlier, P., Marchand- Brynaert, J. Thrombin Inhibitors Designed for Grafting on Biomaterials. Org. Biomol. Chem. v3 pp. 4209, 2005 | |
| 71 | 1D3P | 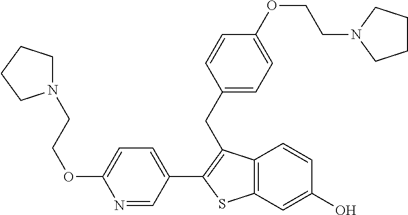 | Chirgadze, N. Y., Sall, D. J., Briggs, S. L., Clawson, D. K., Zhang, M., Smith, G. F., Schevitz, R. W. The crystal structures of human alpha-thrombin complexed with active site-directed diamino benzo[b]thiophene derivatives: a binding mode for a structurally novel class of inhibitors. Protein Sci. v9 pp. 29-36 , 2000 | |
| 72 | 1D3Q | 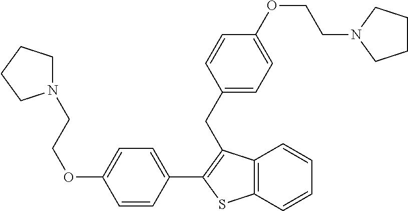 | Chirgadze, N. Y., Sall, D. J., Briggs, S. L., Clawson, D. K., Zhang, M., Smith, G. F., Schevitz, R. W. The crystal structures of human alpha-thrombin complexed with active site-directed diamino benzo[b]thiophene derivatives: a binding mode for a structurally novel class of inhibitors. Protein Sci. v9 pp. 29-36, 2000 | |
| 73 | 1D3T | 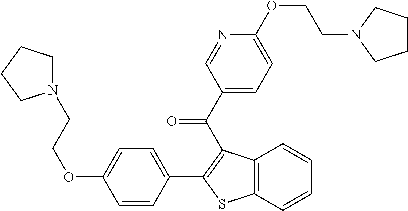 | Chirgadze, N. Y., Sall, D. J., Briggs, S. L., Clawson, D. K., Zhang, M., Smith, G. F., Schevitz, R. W. The crystal structures of human alpha-thrombin complexed with active site-directed diamino benzo[b]thiophene derivatives: a binding mode for a structurally novel class of inhibitors. Protein Sci. v9 pp. 29-36, 2000 | |
| 74 | 1D4P | 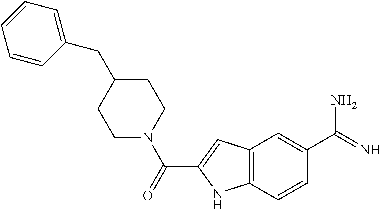 | Chirgadze, N. Y., Sall, D. J., Klimkowski, V. J., Clawson, D. K., Briggs, S. L., Hermann, R., Smith, G. F., Gifford-Moore, D. S., Wery, J. P. The crystal structure of human alpha- thrombin complexed with LY178550, a nonpeptidyl, active site-directed inhibitor. Protein Sci. v6 pp. 1412- 1417, 1997 | |
| 75 | 3HAT | 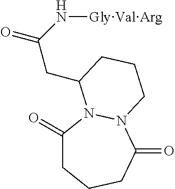 | Mathews, I. I., Tulinsky, A. Active Site Mimetic Inhibition of Thrombin To be Published | |
| 76 | 1DWD | 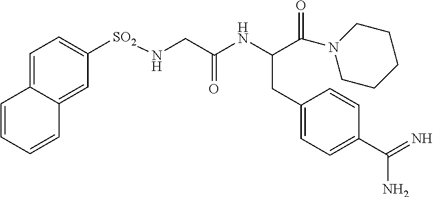 | Banner, D. W., Hadvary, P. Crystallographic analysis at 3.0-A resolution of the binding to human thrombin of four active site-directed inhibitors. J. Biol. Chem. v266 pp. 20085-20093, 1991 | |
| 77 | 1FPC | 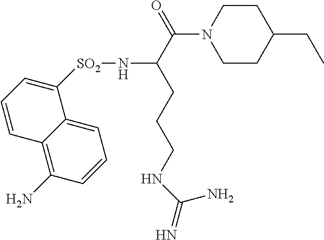 | Mathews, I. I., Tulinsky, A. ACTIVE-SITE MIMETIC INHEBITION OF THROMBIN. Acta Crystallogr D Biol Crystallogr v51 pp. 550- 559, 1995 | |
| 78 | 1TBZ | 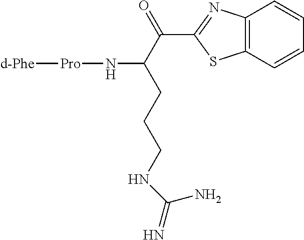 | Matthews, J. H., Krishnan, R., Costanzo, M. J., Maryanoff, B. E., Tulinsky, A. Crystal structures of thrombin with thiazole-containing inhibitors: probes of the S1′ binding site. Biophys. J. v71 pp. 2830- 2839, 1996 | |
| 79 | 1D3D | 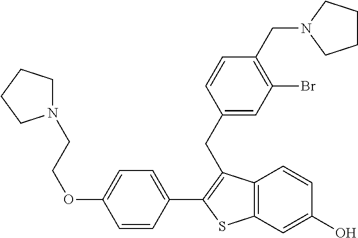 | Chirgadze, N. Y., Sall, D. J., Briggs, S. L., Clawson, D. K., Zhang, M., Smith, G. F., Schevitz, R. W. The crystal structures of human alpha-thrombin complexed with active site-directed diamino benzo[b]thiophene derivatives: a binding mode for a structurally novel class of inhibitors Protein Sci. v9 pp. 29-36, 2000 | |
| 80 | 1QJ6 | 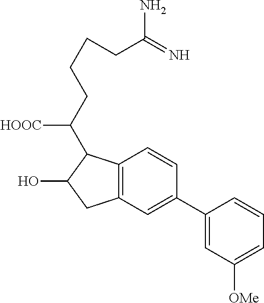 | Jhoti, H., Cleasby, A., Reid, S., Thomas, P. J., Weir, M., Wonacott, A. Crystal structures of thrombin complexed to a novel series of synthetic inhibitors containing a 5,5-trans-lactone template. Biochemistry v38 pp. 7969-7977, 1999 | Covalent inhibitor? |
| 81 | 1QJ7 | 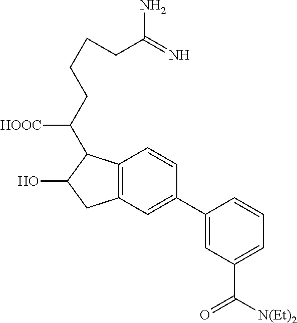 | Jhoti, H., Cleasby, A., Reid, S., Thomas, P. J., Weir, M., Wonacott, A. Crystal structures of thrombin complexed to a novel series of synthetic inhibitors containing a 5,5-trans-lactone template. Biochemistry v38 pp. 7969-7977, 1999 | Covalent inhibitor? |
| 82 | 1UMA | 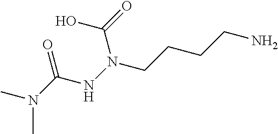 | Nardini, M., Pesce, A., Rizzi, M., Casale, E., Ferraccioli, R., Balliano, G., Milla, P., Ascenzi, P., Bolognesi, M. Human alpha-thrombin inhibition by the active site titrant N alpha- (N,N-dimethylcarbamoyl)- alpha-azalysine p-nitrophenyl ester: a comparative kinetic and X-ray crystallographic study. J. Mol. Biol. v258 pp. 85l-859, 1996 | N,N- DIMETHYL CARBAMOYL ALPHA- AZALYSINE |

TABLE 2
Mass-spectrometric parameters and the computed scoring functions for the thrombin
inhibitors synthesized by the methods described in Examples 1-4

| Nos. | | | Scoring |
|---|---|---|---|
| (Molecular | | Ion mass | function |
| weight) | Chemical formula | (M + 1)⁺ | kcal/mol |
| 1 | 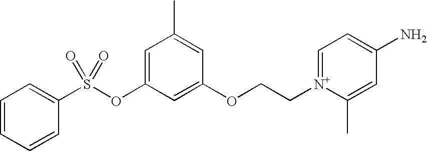 | 399 | −6.51 |
| 2 | 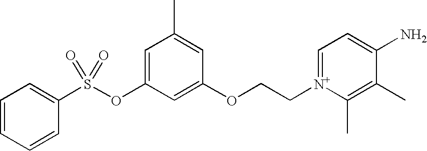 | 413 | −6.60 |
| 3 | 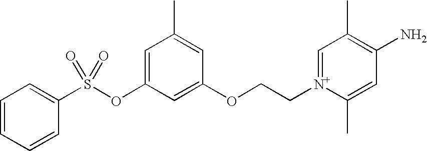 | 413 | −6.42 |
| 4 | 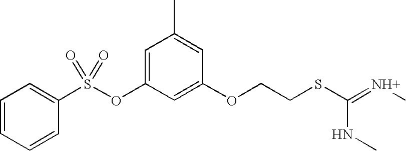 | 383 | −5.51 |
| 5 | 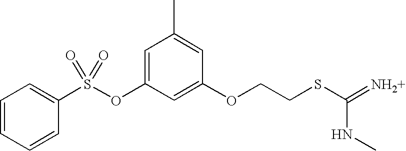 | 369 | −5.86 |
| 6 | 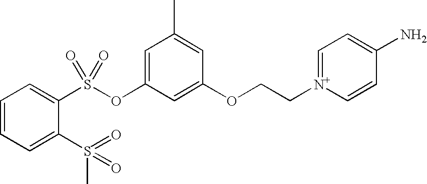 | 463 | −6.60 |
| 7 | 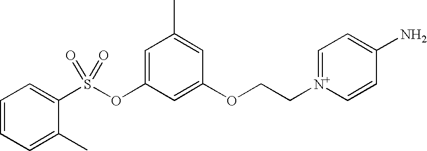 | 399 | −6.81 |
| 8 | 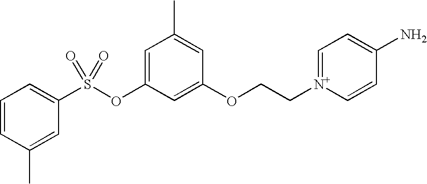 | 399 | −6.92 |
| 9 | 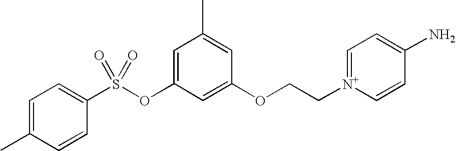 | 399 | −6.75 |
| 10 | 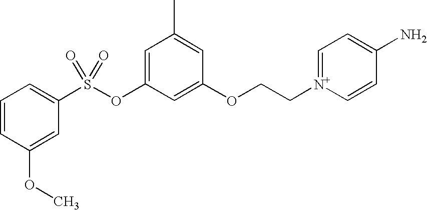 | 415 | −6.93 |
| 11 | 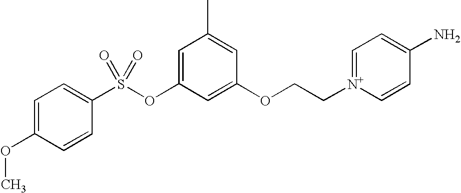 | 415 | −7.02 |
| 12 | 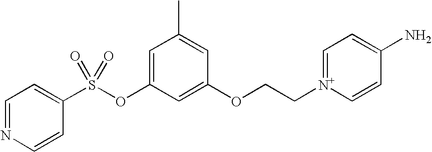 | 386 | −6.73 |
| 13 | 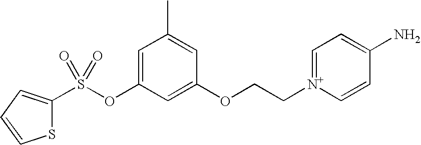 | 391 | −6.92 |
| 14 | 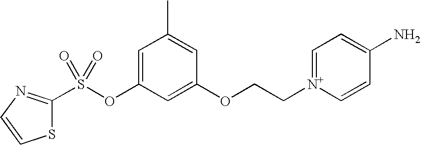 | 392 | −6.45 |
| 15 | 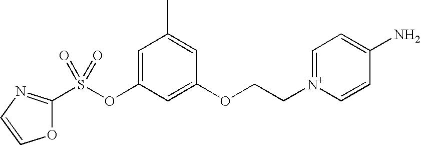 | 376 | −6.21 |
| 16 | 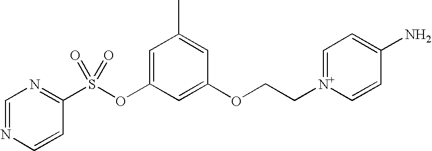 | 387 | −6.45 |
| 17 | 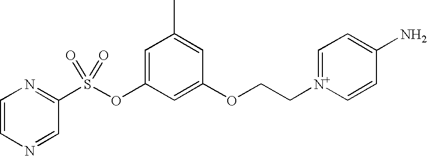 | 387 | −6.51 |
| 18 | 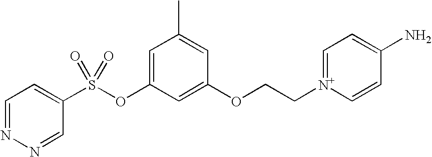 | 387 | −6.43 |
| 19 | 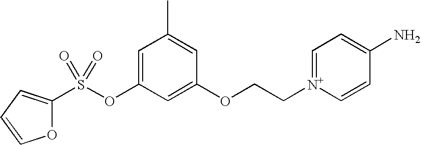 | 375 | −6.67 |
| 20 | 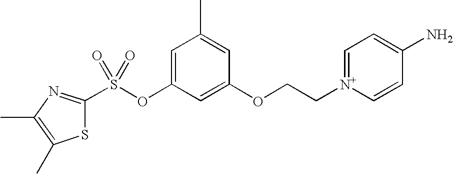 | 420 | −6.93 |
| 21 | 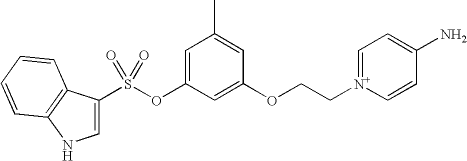 | 424 | −7.23 |
| 22 | 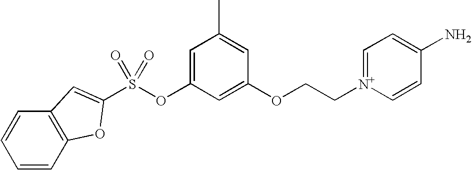 | 425 | −7.12 |
| 23 | 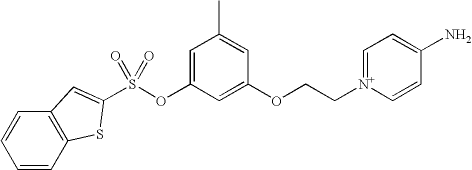 | 441 | −7.43 |
| 24 | 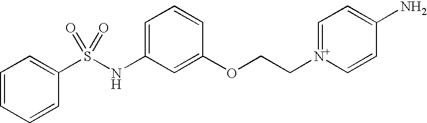 | 370 | −7.01 |
| 25 | 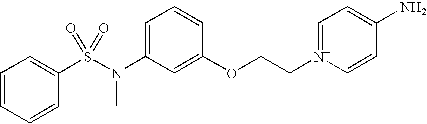 | 384 | −7.04 |
| 26 | 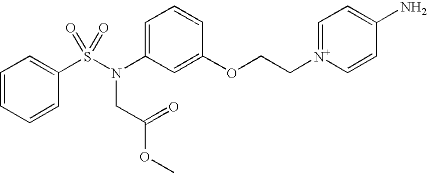 | 442 | −7.12 |
| 27 | 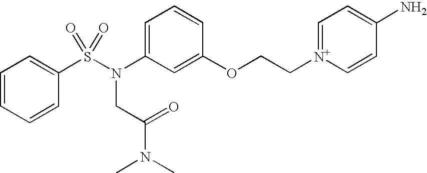 | 455 | −7.15 |
| 28 | 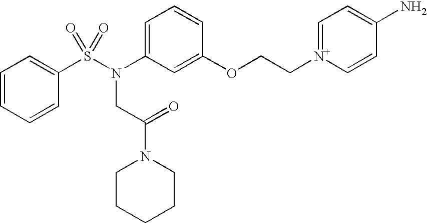 | 495 | −7.21 |
| 29 | 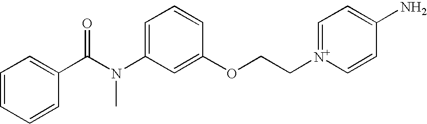 | 348 | −6.23 |
| 30 | 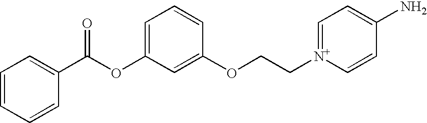 | 335 | −6.13 |
| 31 | 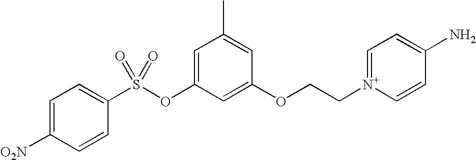 | 430 | −6.56 |
| 32 | 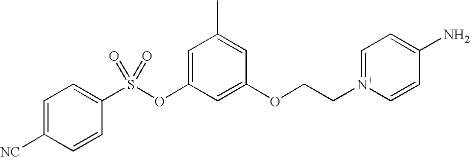 | 410 | −6.71 |
| 33 | 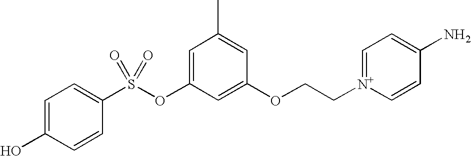 | 401 | −6.33 |
| 34 | 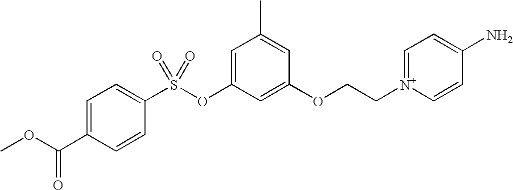 | 443 | −6.84 |
| 35 | 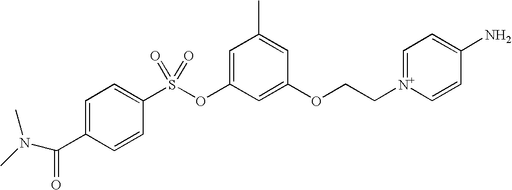 | 456 | −6.82 |
| 36 | 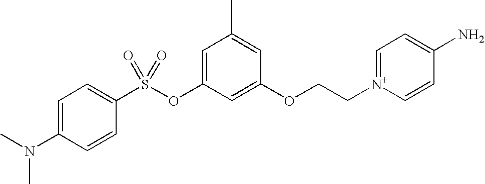 | 428 | −6.51 |
| 37 | 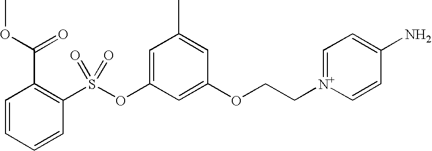 | 443 | −6.92 |
| 38 | 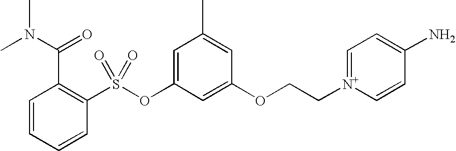 | 456 | −7.12 |
| 39 | 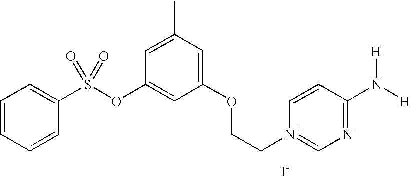 | 386 | −5.45 |

TABLE 3
Mass-spectrometric parameters and the computed scoring functions for the thrombin
inhibitors synthesized by the method described in Example 5

| Nos. | | | Scoring |
|---|---|---|---|
| (Molecular | | Ion mass | function |
| weight) | Chemical formula | (M + 1)⁺ | kcal/mol |
| 1 | 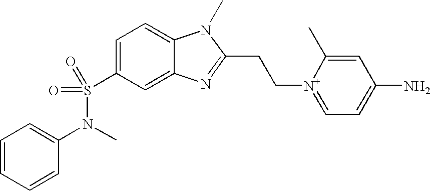 | 436 | −6.63 |
| 2 | 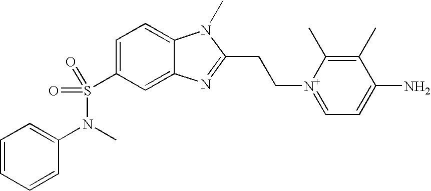 | 450 | −6.41 |
| 3 | 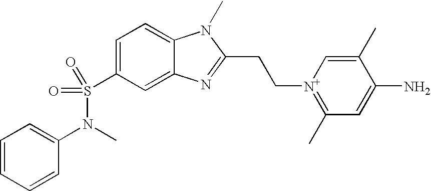 | 450 | −6.45 |
| 4 | 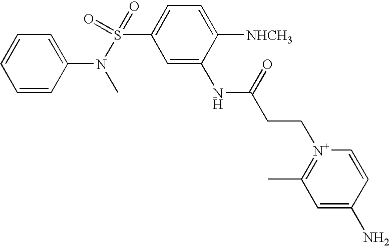 | 454 | −6.83 |
| 5 | 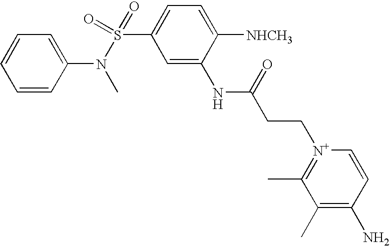 | 468 | −6.54 |
| 6 | 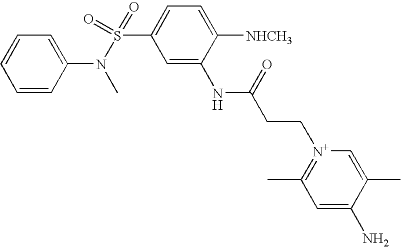 | 468 | −6.42 |
| 7 | 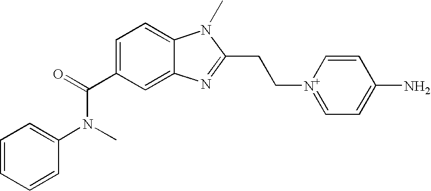 | 386 | −5.93 |
| 8 | 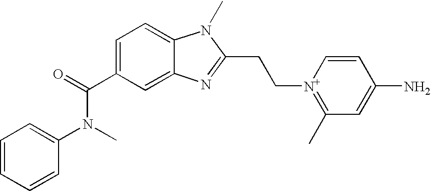 | 400 | −5.63 |
| 9 | 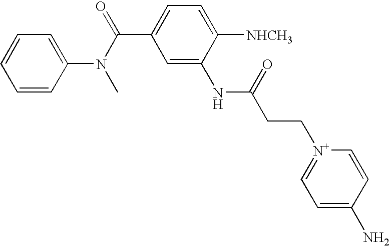 | 404 | −6.21 |

TABLE 4
Examples of variations in the hydrolysis rate of thrombin substrates in the presence of
different concentrations of a series of newly synthesized compounds

| | | Estimate | | | Hydrolysis |
|---|---|---|---|---|---|
| Nos. | | of ΔG | | | rate |
| (Molecular | | binding, | Concentration | | inhibition, |
| weight) | Structural formula of compound | kcal/mol | of compound | | % |
| HC-013s-IOC (M_{B} = 540) | 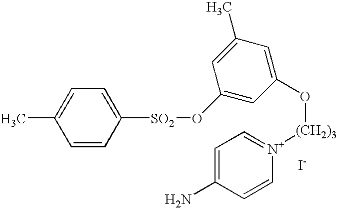 | −6.83 | 0.01 0.02 0.05 0.1 0.25 | mM mM mM mM mM | 11 20 45 65 84 |
| HC-016s-IOC (M_{B} = 526) | 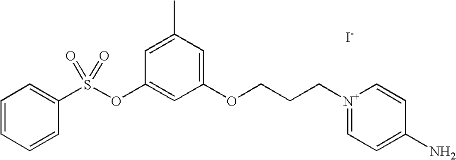 | −6.42 | 0.25 0.5 | mM mM | 84 100 |
| HC-017s-IOC (M_{B} = 532) | 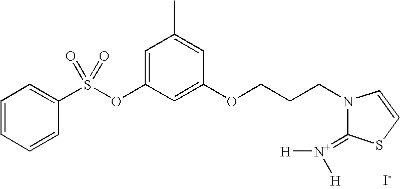 | −5.94 | 100 200 0.5 2 5 20 50 100 200 | nM nM mcM mcM mcM mcM mcM mcM mcM | 5 10 23 57 73 95 95 96 97 |
| HC-018s-IOC (M_{B} = 508) | 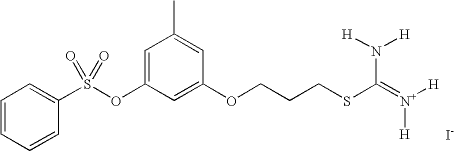 | −5.89 | 20 40 100 200 0.5 1 2 5 10 20 50 100 | nM nM nM nM mcM mcM mcM mcM mcM mcM mcM mcM | 16 33 49 64 93 98 100 100 100 100 100 100 |
| HC-019s-IOC (M_{B} = 512) | 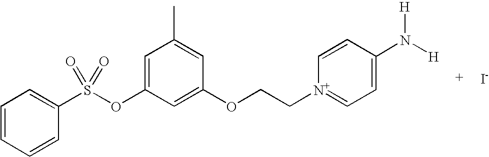 | −6.56 | 2.5 5 12.5 25 50 125 | nM nM nM nM nM nM | 55 88 90 88 95 94 |
| HC-020s-IOC (M_{B} = 494) | 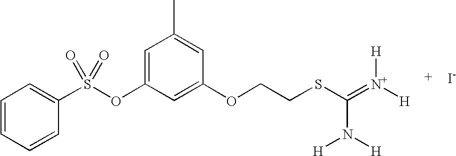 | −6.12 | 5 12.5 25 50 125 250 500 1.25 2.5 5 | nM nM nM nM nM nM nM mcM mcM mcM | 54 46 59 68 81 94 96 98 99 99 |
| HC-021s-IOC (M_{B} = 504.05) | 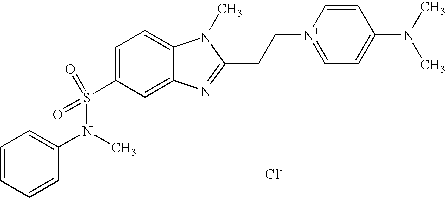 | −5.18 | 25 100 250 500 | mcM mcM mcM mcM | 5 8 4 4 |
| HC-022s-IOC (M_{B} = 486.03) | 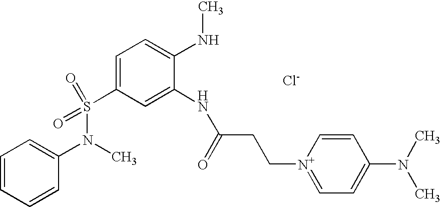 | −5.01 | 0.25 0.5 5 25 50 130 250 | mcM mcM mcM mcM mcM mcM mcM | 21 18 27 34 40 36 51 |
| HC-023s-IOC (M_{B} = 560.5) | 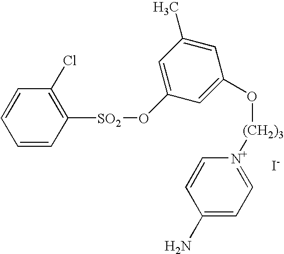 | −6.61 | 0.7 1.4 3.4 34 68 250 | mcM mcM mcM mcM mcM mcM | 13 34 50 86 99 100 |
| HC-024s-IOC (M_{B} = 534.5) | 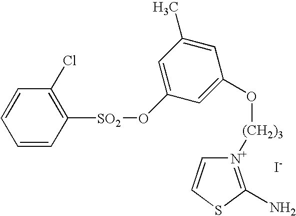 | −5.54 | 0.3 0.68 1.35 3.4 10 | mcM mcM mcM mcM mcM | 46 63 68 82 100 |
| HC-025s-IOC (M_{B} = 546.5) | 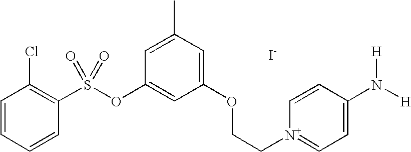 | −6.81 | 1.25 2.5 6.3 12.5 50 125 | nM nM nM nM nM nM | 69 52 70 81 96 98 |
| HC-026s-IOC (M_{B} = 542.5) | 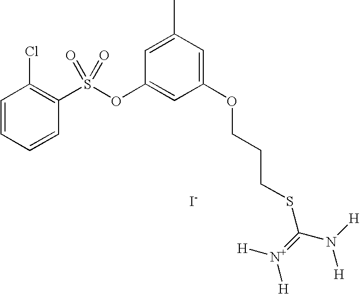 | −5.63 | 5 12.5 25 125 | nM nM nM nM | 47 40 64 68 |
| HC_027s_IOC (Mw = 475) | 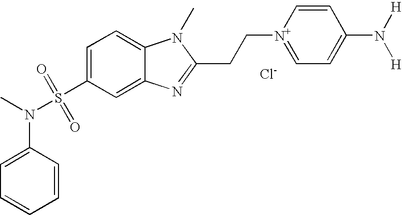 | −6.54 | 0.25 0.5 2.5 5 10 25 50 100 250 500 | mcM mcM mcM mcM mcM mcM mcM mcM mcM mcM | 11 6 24 24 59 72 88 100 100 100 |
| HC_028s_IOC (Mw = 492) | 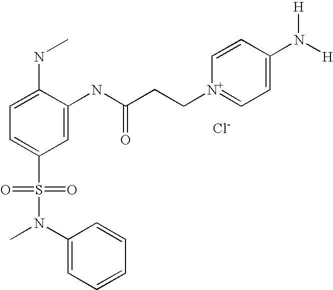 | | 0.1 0.25 0.5 1 2.5 5 25 50 250 500 | mcM mcM mcM mcM mcM mcM mcM mcM mcM mcM | 15 34 46 44 63 78 95 95 100 100 |
| HC_029s_IOC (Mw = 544.5) | 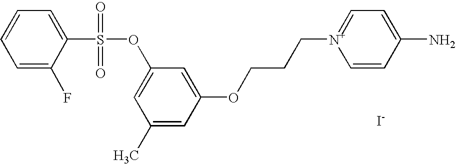 | −5.85 | 50 100 250 0.5 1 2.5 5 | nM nM nM mcM mcM mcM mcM | 8 14 16 25 54 75 81 |
| HC_030s_IOC (Mw = 518.5) | 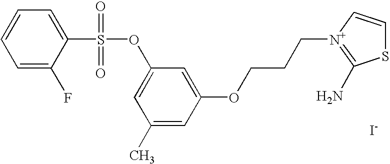 | −6.07 | 20 50 2 5 10 | nM nM mcM mcM mcM | 5 18 72 88 93 |
| HC_031s_IOC (Mw = 526.5) | 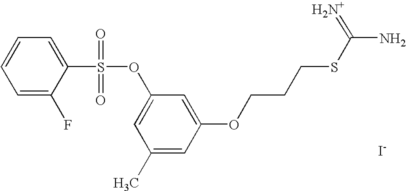 | −5.81 | 5 10 20 50 100 2 5 | nM nM nM nM nM mcM mcM | 35 43 49 59 73 99 100 |
| HC_032s_IOC (Mw = 555.5) | 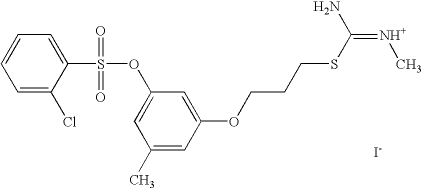 | −5.42 | 50 100 5 10 | nM nM mcM mcM | 10 17 48 71 |
| HC_033s_IOC (Mw = 541.5) | 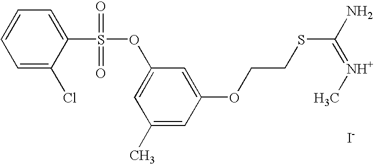 | −5.61 | 4 10 20 40 100 200 4 | nM nM nM nM nM nM mcM | 18 23 24 62 59 74 100 |
| HC_036s_IOC (Mw = 526) | 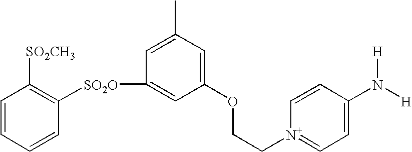 | −6.6 | 2.5 5 25 50 2.5 | nM nM nM nM mcM | 29 28 79 88 96 |
| HC_037s_IOC (Mw = 530.35) | 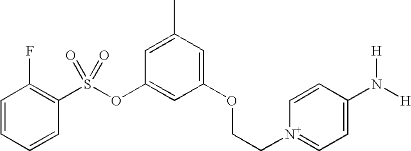 | −6.49 | 2.5 5 25 50 2.5 | nM nM nM nM mcM | 43 59 82 86 89 |
| HC_038s_IOC (Mw = 526.39) | 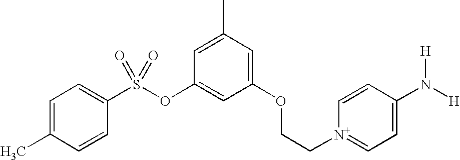 | −6.75 | 5 25 50 2.5 | nM nM nM mcM | 47 56 85 96 |
| HC_039s_IOC (Mw = 546.81) | 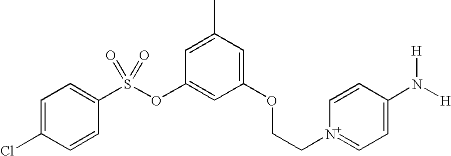 | −7.03 | 2.5 5 25 50 2.5 | nM nM nM nM mcM | 24 44 73 88 98 |
| HC_040s_IOC (Mw = 572.46) | 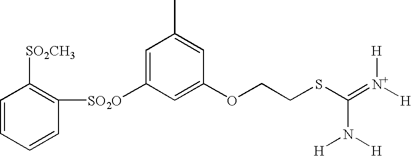 | −5.48 | 2.5 5 25 50 2.5 | nM nM nM nM mcM | 4 19 66 75 100 |
| HC_041s_IOC (Mw = 405.5) | 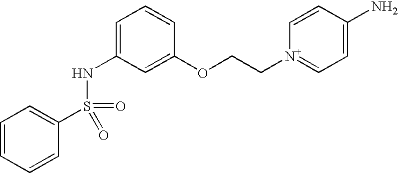 | −7.01 | 0.1 0.25 0.5 1.75 3.75 25 250 | mcM mcM mcM mcM mcM mcM mcM | 56 62 75 88 90 95 99 |
| HC_045s_IOC (Mw = 520.5) | 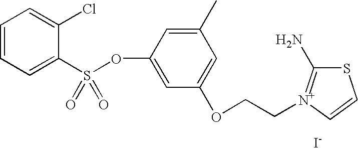 | −5.88 | 0.125 0.25 0.5 1.25 2.5 | mcM mcM mcM mcM mcM | 10 18 42 66 87 |
| HC_046s_IOC (Mw = 528.5) | 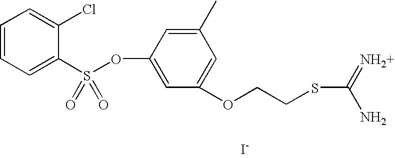 | −6.02 | 1.25 2.5 5 12.5 25 | nM nM nM nM nM | 18 39 59 77 92 |
| HC_047s_IOC (Mw = 513.35) | 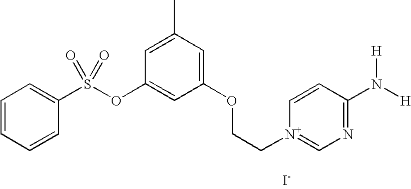 | −5.45 | 10 25 50 0.25 0.5 1.85 2.5 5 | nM nM nM mcM mcM mcM mcM mcM | 8 10 14 36 49 84 90 |

What is claimed is:

1. A compound of the general structural formula (I) and its pharmaceutically acceptable salts or solvates:
A-B-C (I)
wherein C is chosen from a group comprising the structures: 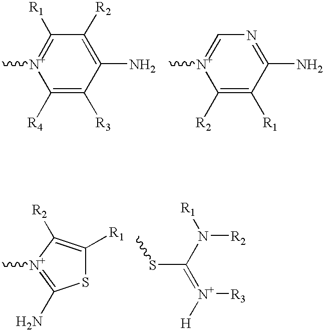
wherein R₁, R₂, R₃, and R₄ independently from one another are hydrogen or C₁₋₆ alkyl;
B is —(CH₂)ₙ—, wherein n is an integer from 1 to 5;
A is chosen from a group comprising the structures: 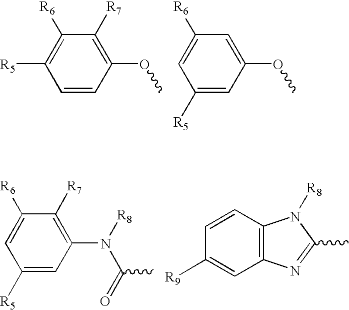
wherein R₅ is chosen from a group comprising hydrogen, C₁₋₆ alkoxy, CH₂NR₁₀R₁₁, and CH(CH₃)NR₁₀R₁₁; 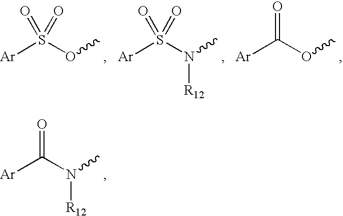
wherein R₆ and R₇ are independently hydrogen, C₁₋₆ alkyl; C₁₋₆ alkoxy; and halogen;
R₈ is hydrogen or C₁₋₆ alkyl;
R₉ is chosen from the following group comprising: 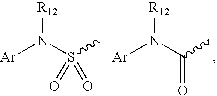
R₁₀ and R₁₂ are independently from each other chosen from a group comprising hydrogen, C₁₋₆ alkyl; (CH₂)ₘCOOR₁₃, and (CH₂)ₘCON(R₁₃)₂, 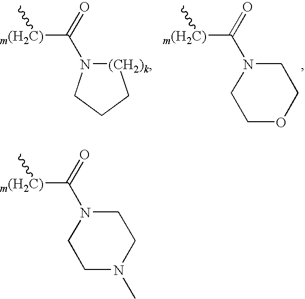
wherein m is an integer from 1 to 4,
R₁₃ is hydrogen or C₁₋₆ alkyl,
R₁₁ is C₁₋₆ alkyl or Ar;
Ar is phenyl, pyridyl, oxazolyl, thiazolyl, thienyl, furanyl, pyrimidinyl, pyridazonyl, pyrazinyl, indolyl, benzofuranyl, or benzothiophenyl having from one to five substituents selected from the group of
hydrogen, C₁₋₆ alkyl, C₁₋₆ alkoxy, halogen, N(R₁₃)₂, OH, NO₂, CN, COOR₁₃, CON(R₁₃)₂, and SO₂R₁₃;
with the exception of:

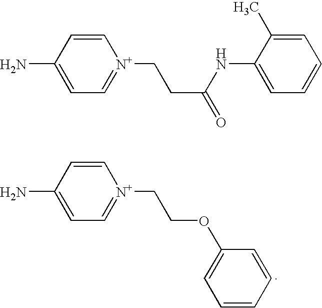

2. A compound of claim 1, and its pharmaceutically acceptable salts or solvates, in particular: 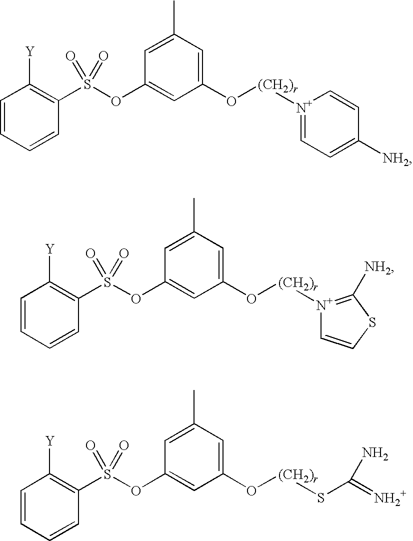
wherein Y is chosen from a group c omprising hydrogen, halogen, COOR₁₃, CON(R₁₃)₂, and SO₂R₁₃; and
r is an integer from 2 to 5.

3. A compound of claim 1, and its pharmaceutically acceptable salts or solvates that are capable to inhibiting thrombin.

4. Application of a compound of claim 1, and its pharmaceutically acceptable salts or solvates as thrombin inhibitors.

5. A pharmaceutical composition for use in treatment and prophylaxis of thrombin-dependent thromboembolic events, comprising a therapeutically effective quantity of a compound of claim 1, its pharmaceutically acceptable salts or solvates, and a pharmaceutically acceptable carrier.
